SEQUENCE LISTING (353 sequences)

SEQ ID NO: 1 - protein, 29 aa, synthetic construct
  VARIANT 2: note = L, R or I
  VARIANT 3: note = R, I, L, V, K, Q, S, H, or A
  VARIANT 4: note = A, R, N, D, Q, E, H, K, M, S, T, W, F or V
  VARIANT 7: note = A, R, D, Q, E, G, K or S
  VARIANT 11: note = A, R, N, D, E, G, K, S, T or Q
  VARIANT 16: note = N or T
  VARIANT 17: note = R or K
  VARIANT 18: note = A, R, N, D, C, Q, E, G, L, K, M, S, T, W or V
  VARIANT 25: note = K, R, Q, H, S, G or A
  VARIANT 26: note = S or K
  VARIANT 28: note = V, R, I, L or A
  VARIANT 29: note = D or K
EXXXAAXEIDXLPNLXXXQWNAFIXXLXX

SEQ ID NO: 2 - protein, 58 aa, synthetic construct
  VARIANT 9: note = L, R or I
  VARIANT 10: note = R, I, L, V, K, Q, S, H, or A
  VARIANT 11: note = A, R, N, D, Q, E, H, K, M, S, T, W, F or V
  VARIANT 14: note = A, R, D, Q, E, G, K or S
  VARIANT 18: note = A, R, N, D, E, G, K, S, T or Q
  VARIANT 23: note = N or T
  VARIANT 24: note = R or K
  VARIANT 25: note = A, R, N, D, C, Q, E, G, L, K, M, S, T, W or V
  VARIANT 32: note = K, R, Q, H, S, G or A
  VARIANT 33: note = S or K
  VARIANT 35: note = V, R, I, L or A
  VARIANT 36: note = D or K
VDNKFNKEXXXAAXEIDXLPNLXXXQWNAFIXXLXXDPSQSANLLAEAKKLNDAQAPK

SEQ ID NO: 3 - protein, 59 aa, synthetic construct
  VARIANT 9: note = L, R or I
  VARIANT 10: note = R, I, L, V, K, Q, S, H, or A
  VARIANT 11: note = A, R, N, D, Q, E, H, K, M, S, T, W, F or V
  VARIANT 14: note = A, R, D, Q, E, G, K or S
  VARIANT 18: note = A, R, N, D, E, G, K, S, T or Q
  VARIANT 23: note = N or T
  VARIANT 24: note = R or K
  VARIANT 25: note = A, R, N, D, C, Q, E, G, L, K, M, S, T, W or V
  VARIANT 32: note = K, R, Q, H, S, G or A
  VARIANT 33: note = S or K
  VARIANT 35: note = V, R, I, L or A
  VARIANT 36: note = D or K
AENKFNKEXXXAAXEIDXLPNLXXXQWNAFIXXLXXDPSQSANLLAEAKKLNDAQAPKC

SEQ ID NO: 4 - protein, 58 aa, synthetic construct
VDNKFNKERAEAAQEIDQLPNLNRGQWNAFIASLVDDPSQSANLLAEAKKLNDAQAPK

SEQ ID NO: 5 - protein, 58 aa, synthetic construct
VDNKFNKERQVAAAEIDALPNLNRGQWNAFIASLVDDPSQSANLLAEAKKLNDAQAPK

SEQ ID NO: 6 - protein, 58 aa, synthetic construct
VDNKFNKELSDAAQEIDSLPNLNRSQWNAFIKSLIDDPSQSANLLAEAKKLNDAQAPK

SEQ ID NO: 7 - protein, 58 aa, synthetic construct
VDNKFNKERREAAAEIDNLPNLNRSQWNAFIQSLVDDPSQSANLLAEAKKLNDAQAPK

SEQ ID NO: 8 - protein, 58 aa, synthetic construct
VDNKFNKERREAAKEIDSLPNLNRTQWNAFIRSLADDPSQSANLLAEAKKLNDAQAPK

SEQ ID NO: 9 - protein, 58 aa, synthetic construct
VDNKFNKELRHAASEIDDLPNLNRAQWNAFIRSLRDDPSQSANLLAEAKKLNDAQAPK

SEQ ID NO: 10 - protein, 58 aa, synthetic construct
VDNKFNKELVRAAQEIDELPNLNRGQWNAFIKSLVDDPSQSANLLAEAKKLNDAQAPK

SEQ ID NO: 11 - protein, 58 aa, synthetic construct
VDNKFNKEIKQAAREIDALPNLNKKQWNAFIQSLADDPSQSANLLAEAKKLNDAQAPK

SEQ ID NO: 12 - protein, 58 aa, synthetic construct
VDNKFNKERHRAAQEIDQLPNLNRRQWNAFIKSLVDDPSQSANLLAEAKKLNDAQAPK

SEQ ID NO: 13 - protein, 58 aa, synthetic construct
VDNKFNKEIKFAAGEIDNLPNLNRKQWNAFIGSLRDDPSQSANLLAEAKKLNDAQAPK

SEQ ID NO: 14 - protein, 58 aa, synthetic construct
VDNKFNKERLKAAAEIDALPNLNRKQWNAFISSLRDDPSQSANLLAEAKKLNDAQAPK

SEQ ID NO: 15 - protein, 58 aa, synthetic construct
VDNKFNKELRAAAAEIDSLPNLNRKQWNAFIKSLVDDPSQSANLLAEAKKLNDAQAPK

SEQ ID NO: 16 - protein, 58 aa, synthetic construct
VDNKFNKERLEAAEEIDSLPNLNRAQWNAFIKSLVDDPSQSANLLAEAKKLNDAQAPK

SEQ ID NO: 17 - protein, 58 aa, synthetic construct
VDNKFNKELIKAAAEIDALPNLNRRQWNAFIRSLVDDPSQSANLLAEAKKLNDAQAPK

SEQ ID NO: 18 - protein, 58 aa, synthetic construct
VDNKFNKERIHAAREIDALPNLNRKQWNAFIKSLVDDPSQSANLLAEAKKLNDAQAPK

SEQ ID NO: 19 - protein, 58 aa, synthetic construct
VDNKFNKELIKAAAEIDGLPNLNRKQWNAFIKSLVDDPSQSANLLAEAKKLNDAQAPK

SEQ ID NO: 20 - protein, 58 aa, synthetic construct
VDNKFNKELVEAAREIDSLPNLNRRQWNAFIKSLVDDPSQSANLLAEAKKLNDAQAPK

SEQ ID NO: 21 - protein, 58 aa, synthetic construct
VDNKFNKELIKAAREIDELPNLNRRQWNAFIKSLVDDPSQSANLLAEAKKLNDAQAPK

SEQ ID NO: 22 - protein, 58 aa, synthetic construct
VDNKFNKELLAAAAEIDSLPNLNRAQWNAFIKSLVDDPSQSANLLAEAKKLNDAQAPK

SEQ ID NO: 23 - protein, 58 aa, synthetic construct
VDNKFNKELINAAKEIDDLPNLNRRQWNAFIKSLVDDPSQSANLLAEAKKLNDAQAPK

SEQ ID NO: 24 - protein, 58 aa, synthetic construct
VDNKFNKELVEAAREIDALPNLNRKQWNAFIKSLVDDPSQSANLLAEAKKLNDAQAPK

SEQ ID NO: 25 - protein, 58 aa, synthetic construct
VDNKFNKELRDAAAEIDRLPNLNRRQWNAFIKSLVDDPSQSANLLAEAKKLNDAQAPK

SEQ ID NO: 26 - protein, 58 aa, synthetic construct
VDNKFNKELSAAAREIDNLPNLNRRQWNAFIKSLVDDPSQSANLLAEAKKLNDAQAPK

SEQ ID NO: 27 - protein, 58 aa, synthetic construct
VDNKFNKELISAAEEIDDLPNLNRRQWNAFIKSLVDDPSQSANLLAEAKKLNDAQAPK

SEQ ID NO: 28 - protein, 58 aa, synthetic construct
VDNKFNKELVDAAREIDELPNLNRVQWNAFIKSLVDDPSQSANLLAEAKKLNDAQAPK

SEQ ID NO: 29 - protein, 58 aa, synthetic construct
VDNKFNKELIEAAREIDALPNLNRSQWNAFIKSLRDDPSQSANLLAEAKKLNDAQAPK

SEQ ID NO: 30 - protein, 58 aa, synthetic construct
VDNKFNKELVEAAKEIDKLPNLNRRQWNAFIKSLVDDPSQSANLLAEAKKLNDAQAPK

SEQ ID NO: 31 - protein, 58 aa, synthetic construct
VDNKFNKELRQAAKEIDNLPNLNRAQWNAFIKSLVDDPSQSANLLAEAKKLNDAQAPK

SEQ ID NO: 32 - protein, 58 aa, synthetic construct
VDNKFNKELVAAAREIDSLPNLNRTQWNAFIKSLVDDPSQSANLLAEAKKLNDAQAPK

SEQ ID NO: 33 - protein, 58 aa, synthetic construct
VDNKFNKELRNAAKEIDSLPNLNRRQWNAFIRSLVDDPSQSANLLAEAKKLNDAQAPK

SEQ ID NO: 34 - protein, 58 aa, synthetic construct
VDNKFNKELVQAAREIDELPNLNRTQWNAFIKSLVDDPSQSANLLAEAKKLNDAQAPK

SEQ ID NO: 35 - protein, 58 aa, synthetic construct
VDNKFNKELREAAEEIDNLPNLNRVQWNAFIKSLVDDPSQSANLLAEAKKLNDAQAPK

SEQ ID NO: 36 - protein, 58 aa, Synthetic construct
VDNKFNKELIEAAREIDNLPNLNRAQWNAFIKSLVDDPSQSANLLAEAKKLNDAQAPK

SEQ ID NO: 37 - protein, 58 aa, synthetic construct
VDNKFNKELINAAREIDGLPNLNRMQWNAFIRSLVDDPSQSANLLAEAKKLNDAQAPK

SEQ ID NO: 38 - protein, 58 aa, synthetic construct
VDNKFNKERIAAAQEIDGLPNLNRRQWNAFIKSLVDDPSQSANLLAEAKKLNDAQAPK

SEQ ID NO: 39 - protein, 58 aa, synthetic construct; identical to SEQ ID NO: 23

SEQ ID NO: 40 - protein, 58 aa, synthetic construct
VDNKFNKERSHAAQEIDALPNLNRVQWNAFIKSLVDDPSQSANLLAEAKKLNDAQAPK

SEQ ID NO: 41 - protein, 58 aa, synthetic construct
VDNKFNKELIAAAKEIDELPNLNRRQWNAFIKSLVDDPSQSANLLAEAKKLNDAQAPK

SEQ ID NO: 42 - protein, 58 aa, synthetic construct
VDNKFNKERIRAAEEIDGLPNLNRWQWNAFIKSLVDDPSQSANLLAEAKKLNDAQAPK

SEQ ID NO: 43 - protein, 58 aa, synthetic construct
VDNKFNKELVQAAREIDALPNLNRQQWNAFIKSLVDDPSQSANLLAEAKKLNDAQAPK

SEQ ID NO: 44 - protein, 58 aa, synthetic construct
VDNKFNKERREAAREIDNLPNLNRRQWNAFIKSLVDDPSQSANLLAEAKKLNDAQAPK

SEQ ID NO: 45 - protein, 58 aa, synthetic construct
VDNKFNKELRWAAGEIDKLPNLNRKQWNAFIKSLVDDPSQSANLLAEAKKLNDAQAPK

SEQ ID NO: 46 - protein, 58 aa, synthetic construct
VDNKFNKELSRAAEEIDRLPNLNRVQWNAFIKSLVDDPSQSANLLAEAKKLNDAQAPK

SEQ ID NO: 47 - protein, 58 aa, synthetic construct
VDNKFNKELIEAAREIDELPNLNRRQWNAFIKSLVDDPSQSANLLAEAKKLNDAQAPK

SEQ ID NO: 48 - protein, 58 aa, synthetic construct
VDNKFNKELIAAAKEIDDLPNLNREQWNAFIKSLVDDPSQSANLLAEAKKLNDAQAPK

SEQ ID NO: 49 - protein, 58 aa, synthetic construct
VDNKFNKERVRAAEEIDSLPNLNRQQWNAFIKSLVDDPSQSANLLAEAKKLNDAQAPK

SEQ ID NO: 50 - protein, 58 aa, synthetic construct
VDNKFNKELIDAAAEIDKLPNLNRGQWNAFIKSLVDDPSQSANLLAEAKKLNDAQAPK

SEQ ID NO: 51 - protein, 58 aa, synthetic construct
VDNKFNKELIDAAAEIDKLPNLNRSQWNAFIKSLVDDPSQSANLLAEAKKLNDAQAPK

SEQ ID NO: 52 - protein, 58 aa, synthetic construct
VDNKFNKELIEAAEEIDRLPNLNRQQWNAFIKSLVDDPSQSANLLAEAKKLNDAQAPK

SEQ ID NO: 53 - protein, 58 aa, synthetic construct
VDNKFNKELIEAAREIDELPNLNRKQWNAFIKSLVDDPSQSANLLAEAKKLNDAQAPK

SEQ ID NO: 54 - protein, 58 aa, synthetic construct
VDNKFNKELREAAEEIDSLPNLNRKQWNAFIKSLVDDPSQSANLLAEAKKLNDAQAPK

SEQ ID NO: 55 - protein, 58 aa, synthetic construct
VDNKFNKELVDAAREIDDLPNLNRGQWNAFIKSLVDDPSQSANLLAEAKKLNDAQAPK

SEQ ID NO: 56 - protein, 58 aa, synthetic construct
VDNKFNKELREAAAEIDALPNLNRKQWNAFIKSLVDDPSQSANLLAEAKKLNDAQAPK

SEQ ID NO: 57 - protein, 58 aa, synthetic construct
VDNKFNKELIEAAAEIDKLPNLNRAQWNAFIKSLVDDPSQSANLLAEAKKLNDAQAPK

SEQ ID NO: 58 - protein, 58 aa, synthetic construct
VDNKFNKELREAAGEIDRLPNLNRRQWNAFIKSLVDDPSQSANLLAEAKKLNDAQAPK

SEQ ID NO: 59 - protein, 58 aa, synthetic construct
VDNKFNKELVRAAEEIDALPNLNRKQWNAFIKSLVDDPSQSANLLAEAKKLNDAQAPK

SEQ ID NO: 60 - protein, 58 aa, synthetic construct
VDNKFNKERSRAAAEIDALPNLNRRQWNAFIKSLVDDPSQSANLLAEAKKLNDAQAPK

SEQ ID NO: 61 - protein, 58 aa, synthetic construct
VDNKFNKELIRAASEIDKLPNLNRRQWNAFIKSLRDDPSQSANLLAEAKKLNDAQAPK

SEQ ID NO: 62 - protein, 58 aa, synthetic construct
VDNKFNKELIKAAQEIDRLPNLNRTQWNAFIRSLVDDPSQSANLLAEAKKLNDAQAPK

SEQ ID NO: 63 - protein, 58 aa, synthetic construct
VDNKFNKELIEAAAEIDALPNLNRRQWNAFIKSLVDDPSQSANLLAEAKKLNDAQAPK

SEQ ID NO: 64 - protein, 58 aa, synthetic construct
VDNKFNKELIWAAGEIDKLPNLNRRQWNAFIKSLVDDPSQSANLLAEAKKLNDAQAPK

SEQ ID NO: 65 - protein, 58 aa, synthetic construct
VDNKFNKERLAAAEEIDNLPNLNRRQWNAFIKSLVDDPSQSANLLAEAKKLNDAQAPK

SEQ ID NO: 66 - protein, 58 aa, synthetic construct
VDNKFNKELRKAAEEIDALPNLNRKQWNAFIKSLVDDPSQSANLLAEAKKLNDAQAPK

SEQ ID NO: 67 - protein, 58 aa, synthetic construct
VDNKFNKELIAAAREIDSLPNLNRRQWNAFIKSLVDDPSQSANLLAEAKKLNDAQAPK

SEQ ID NO: 68 - protein, 58 aa, synthetic construct
VDNKFNKERVKAAEEIDKLPNLNRRQWNAFIKSLVDDPSQSANLLAEAKKLNDAQAPK

SEQ ID NO: 69 - protein, 58 aa, synthetic construct
VDNKFNKELIHAAEEIDRLPNLNRNQWNAFIKSLVDDPSQSANLLAEAKKLNDAQAPK

SEQ ID NO: 70 - protein, 58 aa, synthetic construct
VDNKFNKELINAAGEIDKLPNLNRKQWNAFIKSLVDDPSQSANLLAEAKKLNDAQAPK

SEQ ID NO: 71 - protein, 58 aa, synthetic construct
VDNKFNKELIEAAAEIDALPNLNRAQWNAFIKSLVDDPSQSANLLAEAKKLNDAQAPK

SEQ ID NO: 72 - protein, 58 aa, synthetic construct; identical to SEQ ID NO: 67

SEQ ID NO: 73 - protein, 58 aa, synthetic construct
VDNKFNKERVDAAREIDNLPNLNREQWNAFIRSLVDDPSQSANLLAEAKKLNDAQAPK

SEQ ID NO: 74 - protein, 58 aa, synthetic construct
VDNKFNKELRDAAAEIDRLPNLNRKQWNAFIKSLVDDPSQSANLLAEAKKLNDAQAPK

SEQ ID NO: 75 - protein, 58 aa, synthetic construct
VDNKFNKELIAAAAEIDRLPNLNRVQWNAFIKSLVDDPSQSANLLAEAKKLNDAQAPK

SEQ ID NO: 76 - protein, 58 aa, synthetic construct
VDNKFNKELRAAAEEIDKLPNLNRRQWNAFIKSLIDDPSQSANLLAEAKKLNDAQAPK

SEQ ID NO: 77 - protein, 58 aa, synthetic construct
VDNKFNKELIWAAAEIDRLPNLNREQWNAFIRSLVDDPSQSANLLAEAKKLNDAQAPK

SEQ ID NO: 78 - protein, 58 aa, synthetic construct
VDNKFNKELRAAAKEIDNLPNLNRRQWNAFIKSLVDDPSQSANLLAEAKKLNDAQAPK

SEQ ID NO: 79 - protein, 58 aa, synthetic construct
VDNKFNKELVQAAKEIDNLPNLNRSQWNAFIRSLVDDPSQSANLLAEAKKLNDAQAPK

SEQ ID NO: 80 - protein, 58 aa, synthetic construct
VDNKFNKELIEAAGEIDALPNLNRRQWNAFIKSLVDDPSQSANLLAEAKKLNDAQAPK

SEQ ID NO: 81 - protein, 58 aa, synthetic construct
VDNKFNKELVKAAAEIDALPNLNRRQWNAFIKSLVDDPSQSANLLAEAKKLNDAQAPK

SEQ ID NO: 82 - protein, 58 aa, synthetic construct
VDNKFNKELREAAREIDSLPNLNRSQWNAFIKSLVDDPSQSANLLAEAKKLNDAQAPK

SEQ ID NO: 83 - protein, 58 aa, synthetic construct
VDNKFNKELLEAAKEIDELPNLNRRQWNAFIKSLVDDPSQSANLLAEAKKLNDAQAPK

SEQ ID NO: 84 - protein, 58 aa, synthetic construct
VDNKFNKELVEAAREIDELPNLNRSQWNAFIKSLVDDPSQSANLLAEAKKLNDAQAPK

SEQ ID NO: 85 - protein, 58 aa, synthetic construct
VDNKFNKELREAAAEIDALPNLNRSQWNAFIKSLVDDPSQSANLLAEAKKLNDAQAPK

SEQ ID NO: 86 - protein, 58 aa, synthetic construct
VDNKFNKELVRAAEEIDRLPNLNRAQWNAFIKSLVDDPSQSANLLAEAKKLNDAQAPK

SEQ ID NO: 87 - protein, 58 aa, synthetic construct
VDNKFNKELRAAAAEIDSLPNLNRGQWNAFIKSLVDDPSQSANLLAEAKKLNDAQAPK

SEQ ID NO: 88 - protein, 58 aa, synthetic construct
VDNKFNKELIRAAREIDELPNLNRMQWNAFIKSLVDDPSQSANLLAEAKKLNDAQAPK

SEQ ID NO: 89 - protein, 58 aa, synthetic construct
VDNKFNKELRDAAREIDSLPNLNRRQWNAFIRSLVDDPSQSANLLAEAKKLNDAQAPK

SEQ ID NO: 90 - protein, 58 aa, synthetic construct
VDNKFNKELREAAREIDELPNLNRRQWNAFIKSLVDDPSQSANLLAEAKKLNDAQAPK

SEQ ID NO: 91 - protein, 58 aa, synthetic construct
VDNKFNKELIRAAEEIDKLPNLNRKQWNAFIKSLVDDPSQSANLLAEAKKLNDAQAPK

SEQ ID NO: 92 - protein, 58 aa, synthetic construct
VDNKFNKELLKAAREIDELPNLNRRQWNAFIRSLVDDPSQSANLLAEAKKLNDAQAPK

SEQ ID NO: 93 - protein, 58 aa, synthetic construct
VDNKFNKELIAAAREIDGLPNLNRKQWNAFIKSLVDDPSQSANLLAEAKKLNDAQAPK

SEQ ID NO: 94 - protein, 58 aa, synthetic construct
VDNKFNKELVAAAAEIDGLPNLNRKQWNAFIHSLVDDPSQSANLLAEAKKLNDAQAPK

SEQ ID NO: 95 - protein, 58 aa, synthetic construct
VDNKFNKELRDAAREIDALPNLNRAQWNAFIKSLVDDPSQSANLLAEAKKLNDAQAPK

SEQ ID NO: 96 - protein, 58 aa, synthetic construct
VDNKFNKERINAAKEIDSLPNLNRRQWNAFIKSLVDDPSQSANLLAEAKKLNDAQAPK

SEQ ID NO: 97 - protein, 58 aa, synthetic construct
VDNKFNKELRQAAAEIDALPNLNRQQWNAFIKSLVDDPSQSANLLAEAKKLNDAQAPK

SEQ ID NO: 98 - protein, 58 aa, synthetic construct
VDNKFNKELVEAAAEIDRLPNLNRGQWNAFIKSLVDDPSQSANLLAEAKKLNDAQAPK

SEQ ID NO: 99 - protein, 58 aa, synthetic construct
VDNKFNKELREAAAEIDRLPNLNRKQWNAFIKSLVDDPSQSANLLAEAKKLNDAQAPK

SEQ ID NO: 100 - protein, 58 aa, synthetic construct
VDNKFNKELIQAAKEIDSLPNLNRRQWNAFIKSLVDDPSQSANLLAEAKKLNDAQAPK

SEQ ID NO: 101 - protein, 58 aa, synthetic construct
VDNKFNKERSMAAKEIDSLPNLNRRQWNAFIKSLVDDPSQSANLLAEAKKLNDAQAPK

SEQ ID NO: 102 - protein, 58 aa, synthetic construct
VDNKFNKELVAAAREIDDLPNLNRKQWNAFIRSLVDDPSQSANLLAEAKKLNDAQAPK

SEQ ID NO: 103 - protein, 58 aa, synthetic construct
VDNKFNKELVRAAEEIDRLPNLNRLQWNAFIKSLIDDPSQSANLLAEAKKLNDAQAPK

SEQ ID NO: 104 - protein, 58 aa, synthetic construct
VDNKFNKELREAAAEIDDLPNLNRAQWNAFIKSLVDDPSQSANLLAEAKKLNDAQAPK

SEQ ID NO: 105 - protein, 58 aa, synthetic construct
VDNKFNKELVEAAKEIDSLPNLNRRQWNAFIKSLVDDPSQSANLLAEAKKLNDAQAPK

SEQ ID NO: 106 - protein, 58 aa, synthetic construct
VDNKFNKELINAAGEIDRLPNLNRRQWNAFIKSLVDDPSQSANLLAEAKKLNDAQAPK

SEQ ID NO: 107 - protein, 58 aa, synthetic construct
VDNKFNKELLSAAAEIDSLPNLNRKQWNAFIKSLVDDPSQSANLLAEAKKLNDAQAPK

SEQ ID NO: 108 - protein, 58 aa, synthetic construct
VDNKFNKELRDAAAEIDKLPNLNRRQWNAFIKSLVDDPSQSANLLAEAKKLNDAQAPK

SEQ ID NO: 109 - protein, 58 aa, synthetic construct
VDNKFNKELRQAAAEIDKLPNLNRSQWNAFIQSLVDDPSQSANLLAEAKKLNDAQAPK

SEQ ID NO: 110 - protein, 58 aa, synthetic construct
VDNKFNKELREAAAEIDALPNLNRVQWNAFIKSLRDDPSQSANLLAEAKKLNDAQAPK

SEQ ID NO: 111 - protein, 58 aa, synthetic construct
VDNKFNKELVAAAEEIDRLPNLNRAQWNAFIKSLVDDPSQSANLLAEAKKLNDAQAPK

SEQ ID NO: 112 - protein, 58 aa, synthetic construct
VDNKFNKELIDAAAEIDKLPNLNRNQWNAFIKSLVDDPSQSANLLAEAKKLNDAQAPK

SEQ ID NO: 113 - protein, 58 aa, synthetic construct
VDNKFNKERINAAAEIDGLPNLNRQQWNAFIKSLVDDPSQSANLLAEAKKLNDAQAPK

SEQ ID NO: 114 - protein, 58 aa, synthetic construct
VDNKFNKELVRAAEEIDNLPNLNRKQWNAFIKSLVDDPSQSANLLAEAKKLNDAQAPK

SEQ ID NO: 115 - protein, 58 aa, synthetic construct
VDNKFNKELIRAAKEIDELPNLNRRQWNAFIRSLVDDPSQSANLLAEAKKLNDAQAPK

SEQ ID NO: 116 - protein, 58 aa, synthetic construct
VDNKFNKELIQAAREIDSLPNLNRRQWNAFIKSLVDDPSQSANLLAEAKKLNDAQAPK

SEQ ID NO: 117 - protein, 58 aa, synthetic construct
VDNKFNKELIDAAREIDDLPNLNRQQWNAFIKSLLDDPSQSANLLAEAKKLNDAQAPK

SEQ ID NO: 118 - protein, 58 aa, synthetic construct
VDNKFNKELLKAADEIDALPNLNRAQWNAFIKSLVDDPSQSANLLAEAKKLNDAQAPK

SEQ ID NO: 119 - protein, 58 aa, synthetic construct
VDNKFNKELLEAAAEIDSLPNLNRRQWNAFIKSLVDDPSQSANLLAEAKKLNDAQAPK

SEQ ID NO: 120 - protein, 58 aa, synthetic construct
VDNKFNKELVDAAREIDTLPNLNRRQWNAFIKSLVDDPSQSANLLAEAKKLNDAQAPK

SEQ ID NO: 121 - protein, 58 aa, synthetic construct
VDNKFNKELIEAAREIDKLPNLNRRQWNAFIRSLVDDPSQSANLLAEAKKLNDAQAPK

SEQ ID NO: 122 - protein, 58 aa, synthetic construct
VDNKFNKELVAAAREIDNLPNLNRRQWNAFIKSLVDDPSQSANLLAEAKKLNDAQAPK

SEQ ID NO: 123 - protein, 58 aa, synthetic construct
VDNKFNKELVEAAAEIDGLPNLNRDQWNAFIKSLRDDPSQSANLLAEAKKLNDAQAPK

SEQ ID NO: 124 - protein, 58 aa, synthetic construct
VDNKFNKELRNAAKEIDGLPNLNRRQWNAFIKSLVDDPSQSANLLAEAKKLNDAQAPK

SEQ ID NO: 125 - protein, 58 aa, synthetic construct
VDNKFNKELRHAAREIDGLPNLNRTQWNAFIKSLVDDPSQSANLLAEAKKLNDAQAPK

SEQ ID NO: 126 - protein, 58 aa, synthetic construct
VDNKFNKELVEAAREIDTLPNLNRRQWNAFIKSLRDDPSQSANLLAEAKKLNDAQAPK

SEQ ID NO: 127 - protein, 58 aa, synthetic construct
VDNKFNKELVAAAAEIDALPNLNRRQWNAFIKSLVDDPSQSANLLAEAKKLNDAQAPK

SEQ ID NO: 128 - protein, 58 aa, synthetic construct
VDNKFNKELRTAAKEIDDLPNLNRKQWNAFIKSLVDDPSQSANLLAEAKKLNDAQAPK

SEQ ID NO: 129 - protein, 58 aa, synthetic construct
VDNKFNKELLRAAREIDSLPNLNRTQWNAFIKSLVDDPSQSANLLAEAKKLNDAQAPK

SEQ ID NO: 130 - protein, 58 aa, synthetic construct
VDNKFNKERIRAAREIDALPNLNRQQWNAFIKSLVDDPSQSANLLAEAKKLNDAQAPK

SEQ ID NO: 131 - protein, 58 aa, synthetic construct
VDNKFNKELRAAAEEIDRLPNLNRRQWNAFIKSLVDDPSQSANLLAEAKKLNDAQAPK

SEQ ID NO: 132 - protein, 58 aa, synthetic construct
VDNKFNKELIEAAAEIDALPNLNRKQWNAFISSLVDDPSQSANLLAEAKKLNDAQAPK

SEQ ID NO: 133 - protein, 58 aa, synthetic construct
VDNKFNKERIEAAREIDELPNLNRKQWNAFISSLVDDPSQSANLLAEAKKLNDAQAPK

SEQ ID NO: 134 - protein, 58 aa, synthetic construct
VDNKFNKELLAAAKEIDELPNLNRAQWNAFIKSLVDDPSQSANLLAEAKKLNDAQAPK

SEQ ID NO: 135 - protein, 58 aa, synthetic construct
VDNKFNKELINAAREIDELPNLNRRQWNAFIKSLVDDPSQSANLLAEAKKLNDAQAPK

SEQ ID NO: 136 - protein, 58 aa, synthetic construct; identical to SEQ ID NO: 81

SEQ ID NO: 137 - protein, 58 aa, synthetic construct; identical to SEQ ID NO: 41

SEQ ID NO: 138 - protein, 58 aa, synthetic construct
VDNKFNKELISAAREIDKLPNLNRQQWNAFIKSLVDDPSQSANLLAEAKKLNDAQAPK

SEQ ID NO: 139 - protein, 58 aa, synthetic construct
VDNKFNKELREAAEEIDKLPNLNRWQWNAFIKSLVDDPSQSANLLAEAKKLNDAQAPK

SEQ ID NO: 140 - protein, 58 aa, synthetic construct
VDNKFNKELVEAAREIDGLPNLNRAQWNAFIKSLVDDPSQSANLLAEAKKLNDAQAPK

SEQ ID NO: 141 - protein, 58 aa, synthetic construct
VDNKFNKELVAAAQEIDNLPNLNRKQWNAFISSLVDDPSQSANLLAEAKKLNDAQAPK

SEQ ID NO: 142 - protein, 58 aa, synthetic construct
VDNKFNKELRNAAAEIDKLPNLNRRQWNAFIKSLVDDPSQSANLLAEAKKLNDAQAPK

SEQ ID NO: 143 - protein, 58 aa, synthetic construct
VDNKFNKELIQAASEIDALPNLNRTQWNAFIKSLVDDPSQSANLLAEAKKLNDAQAPK

SEQ ID NO: 144 - protein, 58 aa, synthetic construct
VDNKFNKELVEAAAEIDSLPNLNRRQWNAFIKSLVDDPSQSANLLAEAKKLNDAQAPK

SEQ ID NO: 145 - protein, 58 aa, synthetic construct
VDNKFNKERIAAAQEIDALPNLNRRQWNAFIRSLVDDPSQSANLLAEAKKLNDAQAPK

SEQ ID NO: 146 - protein, 58 aa, synthetic construct
VDNKFNKELRDAAKEIDSLPNLNRRQWNAFIKSLVDDPSQSANLLAEAKKLNDAQAPK

SEQ ID NO: 147 - protein, 58 aa, synthetic construct
VDNKFNKELVAAAKEIDKLPNLNRKQWNAFIKSLVDDPSQSANLLAEAKKLNDAQAPK

SEQ ID NO: 148 - protein, 58 aa, synthetic construct
VDNKFNKELIKAAREIDELPNLNRKQWNAFIKSLIDDPSQSANLLAEAKKLNDAQAPK

SEQ ID NO: 149 - protein, 58 aa, synthetic construct
VDNKFNKELVEAASEIDGLPNLNRNQWNAFIKSLVDDPSQSANLLAEAKKLNDAQAPK

SEQ ID NO: 150 - protein, 58 aa, synthetic construct
VDNKFNKELLRAAEEIDNLPNLNRAQWNAFIKSLVDDPSQSANLLAEAKKLNDAQAPK

SEQ ID NO: 151 - protein, 58 aa, synthetic construct
VDNKFNKELIKAAKEIDALPNLNRRQWNAFIKSLVDDPSQSANLLAEAKKLNDAQAPK

SEQ ID NO: 152 - protein, 58 aa, synthetic construct
VDNKFNKELRRAAAEIDSLPNLNRDQWNAFIRSLIDDPSQSANLLAEAKKLNDAQAPK

SEQ ID NO: 153 - protein, 58 aa, synthetic construct; identical to SEQ ID NO: 140

SEQ ID NO: 154 - protein, 58 aa, synthetic construct
VDNKFNKELVEAAREIDELPNLNRRQWNAFIRSLVDDPSQSANLLAEAKKLNDAQAPK

SEQ ID NO: 155 - protein, 58 aa, synthetic construct
VDNKFNKELVWAAAEIDRLPNLNRCQWNAFIKSLVDDPSQSANLLAEAKKLNDAQAPK

SEQ ID NO: 156 - protein, 58 aa, synthetic construct
VDNKFNKERIRAAREIDELPNLNREQWNAFIKSLVDDPSQSANLLAEAKKLNDAQAPK

SEQ ID NO: 157 - protein, 58 aa, synthetic construct
VDNKFNKELRQAAKEIDALPNLNRRQWNAFIKSLVDDPSQSANLLAEAKKLNDAQAPK

SEQ ID NO: 158 - protein, 58 aa, synthetic construct
VDNKFNKELREAAAEIDKLPNLNRKQWNAFIRSLVDDPSQSANLLAEAKKLNDAQAPK

SEQ ID NO: 159 - protein, 58 aa, synthetic construct
VDNKFNKELRRAAEEIDKLPNLNRQQWNAFIKSLVDDPSQSANLLAEAKKLNDAQAPK

SEQ ID NO: 160 - protein, 58 aa, synthetic construct; identical to SEQ ID NO: 43

SEQ ID NO: 161 - protein, 58 aa, synthetic construct
VDNKFNKELRAAAAEIDALPNLNRRQWNAFIKSLVDDPSQSANLLAEAKKLNDAQAPK

SEQ ID NO: 162 - protein, 58 aa, synthetic construct
VDNKFNKELRAAAAEIDKLPNLNRKQWNAFIKSLVDDPSQSANLLAEAKKLNDAQAPK

SEQ ID NO: 163 - protein, 58 aa, synthetic construct
VDNKFNKERIQAAKEIDELPNLNREQWNAFIKSLVDDPSQSANLLAEAKKLNDAQAPK

SEQ ID NO: 164 - protein, 58 aa, synthetic construct
VDNKFNKELVAAAEEIDKLPNLNRRQWNAFIKSLVDDPSQSANLLAEAKKLNDAQAPK

SEQ ID NO: 165 - protein, 58 aa, synthetic construct; identical to SEQ ID NO: 114

SEQ ID NO: 166 - protein, 58 aa, synthetic construct
VDNKFNKELRTAAAEIDALPNLNREQWNAFIRSLVDDPSQSANLLAEAKKLNDAQAPK

SEQ ID NO: 167 - protein, 58 aa, synthetic construct
VDNKFNKELVEAAAEIDALPNLNRKQWNAFIKSLVDDPSQSANLLAEAKKLNDAQAPK

SEQ ID NO: 168 - protein, 58 aa, synthetic construct
VDNKFNKELIKAAEEIDKLPNLNRMQWNAFIRSLVDDPSQSANLLAEAKKLNDAQAPK

SEQ ID NO: 169 - protein, 58 aa, synthetic construct
VDNKFNKELREAAAEIDNLPNLNRRQWNAFIKSLIDDPSQSANLLAEAKKLNDAQAPK

SEQ ID NO: 170 - protein, 58 aa, synthetic construct
VDNKFNKELIEAAREIDALPNLNRKQWNAFIRSLVDDPSQSANLLAEAKKLNDAQAPK

SEQ ID NO: 171 - protein, 58 aa, synthetic construct
VDNKFNKELREAAQEIDSLPNLNRQQWNAFIKSLVDDPSQSANLLAEAKKLNDAQAPK

SEQ ID NO: 172 - protein, 58 aa, synthetic construct
VDNKFNKELRSAAEEIDSLPNLNRRQWNAFIKSLVDDPSQSANLLAEAKKLNDAQAPK

SEQ ID NO: 173 - protein, 58 aa, synthetic construct
VDNKFNKELVEAAAEIDNLPNLNRMQWNAFIKSLVDDPSQSANLLAEAKKLNDAQAPK

SEQ ID NO: 174 - protein, 58 aa, synthetic construct
VDNKFNKELVEAAGEIDNLPNLNRRQWNAFIRSLVDDPSQSANLLAEAKKLNDAQAPK

SEQ ID NO: 175 - protein, 58 aa, synthetic construct
VDNKFNKELSKAAAEIDALPNLNRRQWNAFIKSLVDDPSQSANLLAEAKKLNDAQAPK

SEQ ID NO: 176 - protein, 58 aa, synthetic construct
VDNKFNKELLWAAGEIDRLPNLNRQQWNAFIKSLVDDPSQSANLLAEAKKLNDAQAPK

SEQ ID NO: 177 - protein, 58 aa, synthetic construct
VDNKFNKELRQAAAEIDGLPNLNRRQWNAFIKSLVDDPSQSANLLAEAKKLNDAQAPK

SEQ ID NO: 178 - protein, 59 aa, synthetic construct
AENKFNKERAEAAQEIDQLPNLNRGQWNAFIASLVDDPSQSANLLAEAKKLNDAQAPKC

SEQ ID NO: 179 - protein, 59 aa, synthetic construct
AENKFNKERQVAAAEIDALPNLNRGQWNAFIASLVDDPSQSANLLAEAKKLNDAQAPKC

SEQ ID NO: 180 - protein, 59 aa, synthetic construct
AENKFNKELSDAAQEIDSLPNLNRSQWNAFIKSLIDDPSQSANLLAEAKKLNDAQAPKC

SEQ ID NO: 181 - protein, 59 aa, synthetic construct
AENKFNKERREAAAEIDNLPNLNRSQWNAFIQSLVDDPSQSANLLAEAKKLNDAQAPKC

SEQ ID NO: 182 - protein, 59 aa, synthetic construct
AENKFNKERREAAKEIDSLPNLNRTQWNAFIRSLADDPSQSANLLAEAKKLNDAQAPKC

SEQ ID NO: 183 - protein, 59 aa, synthetic construct
AENKFNKELRHAASEIDDLPNLNRAQWNAFIRSLRDDPSQSANLLAEAKKLNDAQAPKC

SEQ ID NO: 184 - protein, 59 aa, synthetic construct
AENKFNKELVRAAQEIDELPNLNRGQWNAFIKSLVDDPSQSANLLAEAKKLNDAQAPKC

SEQ ID NO: 185 - protein, 59 aa, synthetic construct
AENKFNKEIKQAAREIDALPNLNKKQWNAFIQSLADDPSQSANLLAEAKKLNDAQAPKC

SEQ ID NO: 186 - protein, 59 aa, synthetic construct
AENKFNKERHRAAQEIDQLPNLNRRQWNAFIKSLVDDPSQSANLLAEAKKLNDAQAPKC

SEQ ID NO: 187 - protein, 59 aa, synthetic construct
AENKFNKEIKFAAGEIDNLPNLNRKQWNAFIGSLRDDPSQSANLLAEAKKLNDAQAPKC

SEQ ID NO: 188 - protein, 59 aa, synthetic construct
AENKFNKERLKAAAEIDALPNLNRKQWNAFISSLRDDPSQSANLLAEAKKLNDAQAPKC

SEQ ID NO: 189 - protein, 59 aa, synthetic construct
AENKFNKELRAAAAEIDSLPNLNRKQWNAFIKSLVDDPSQSANLLAEAKKLNDAQAPKC

SEQ ID NO: 190 - protein, 59 aa, synthetic construct
AENKFNKERLEAAEEIDSLPNLNRAQWNAFIKSLVDDPSQSANLLAEAKKLNDAQAPKC

SEQ ID NO: 191 - protein, 59 aa, synthetic construct
AENKFNKELIKAAAEIDALPNLNRRQWNAFIRSLVDDPSQSANLLAEAKKLNDAQAPKC

SEQ ID NO: 192 - protein, 59 aa, synthetic construct
AENKFNKERIHAAREIDALPNLNRKQWNAFIKSLVDDPSQSANLLAEAKKLNDAQAPKC

SEQ ID NO: 193 - protein, 59 aa, synthetic construct
AENKFNKELIKAAAEIDGLPNLNRKQWNAFIKSLVDDPSQSANLLAEAKKLNDAQAPKC

SEQ ID NO: 194 - protein, 59 aa, synthetic construct
AENKFNKELVEAAREIDSLPNLNRRQWNAFIKSLVDDPSQSANLLAEAKKLNDAQAPKC

SEQ ID NO: 195 - protein, 59 aa, synthetic construct
AENKFNKELIKAAREIDELPNLNRRQWNAFIKSLVDDPSQSANLLAEAKKLNDAQAPKC

SEQ ID NO: 196 - protein, 59 aa, synthetic construct
AENKFNKELLAAAAEIDSLPNLNRAQWNAFIKSLVDDPSQSANLLAEAKKLNDAQAPKC

SEQ ID NO: 197 - protein, 59 aa, synthetic construct
AENKFNKELINAAKEIDDLPNLNRRQWNAFIKSLVDDPSQSANLLAEAKKLNDAQAPKC

SEQ ID NO: 198 - protein, 59 aa, synthetic construct
AENKFNKELVEAAREIDALPNLNRKQWNAFIKSLVDDPSQSANLLAEAKKLNDAQAPKC

SEQ ID NO: 199 - protein, 59 aa, synthetic construct
AENKFNKELRDAAAEIDRLPNLNRRQWNAFIKSLVDDPSQSANLLAEAKKLNDAQAPKC

SEQ ID NO: 200 - protein, 59 aa, synthetic construct
AENKFNKELSAAAREIDNLPNLNRRQWNAFIKSLVDDPSQSANLLAEAKKLNDAQAPKC

SEQ ID NO: 201 - protein, 59 aa, synthetic construct
AENKFNKELISAAEEIDDLPNLNRRQWNAFIKSLVDDPSQSANLLAEAKKLNDAQAPKC

SEQ ID NO: 202 - protein, 59 aa, synthetic construct
AENKFNKELVDAAREIDELPNLNRVQWNAFIKSLVDDPSQSANLLAEAKKLNDAQAPKC

SEQ ID NO: 203 - protein, 59 aa, synthetic construct
AENKFNKELIEAAREIDALPNLNRSQWNAFIKSLRDDPSQSANLLAEAKKLNDAQAPKC

SEQ ID NO: 204 - protein, 59 aa, synthetic construct
AENKFNKELVEAAKEIDKLPNLNRRQWNAFIKSLVDDPSQSANLLAEAKKLNDAQAPKC

SEQ ID NO: 205 - protein, 59 aa, synthetic construct
AENKFNKELRQAAKEIDNLPNLNRAQWNAFIKSLVDDPSQSANLLAEAKKLNDAQAPKC

SEQ ID NO: 206 - protein, 59 aa, synthetic construct
AENKFNKELVAAAREIDSLPNLNRTQWNAFIKSLVDDPSQSANLLAEAKKLNDAQAPKC

SEQ ID NO: 207 - protein, 59 aa, synthetic construct
AENKFNKELRNAAKEIDSLPNLNRRQWNAFIRSLVDDPSQSANLLAEAKKLNDAQAPKC

SEQ ID NO: 208 - protein, 59 aa, synthetic construct
AENKFNKELVQAAREIDELPNLNRTQWNAFIKSLVDDPSQSANLLAEAKKLNDAQAPKC

SEQ ID NO: 209 - protein, 59 aa, synthetic construct
AENKFNKELREAAEEIDNLPNLNRVQWNAFIKSLVDDPSQSANLLAEAKKLNDAQAPKC

SEQ ID NO: 210 - protein, 59 aa, synthetic construct
AENKFNKELIEAAREIDNLPNLNRAQWNAFIKSLVDDPSQSANLLAEAKKLNDAQAPKC

SEQ ID NO: 211 - protein, 59 aa, synthetic construct
AENKFNKELINAAREIDGLPNLNRMQWNAFIRSLVDDPSQSANLLAEAKKLNDAQAPKC

SEQ ID NO: 212 - protein, 59 aa, synthetic construct
AENKFNKERIAAAQEIDGLPNLNRRQWNAFIKSLVDDPSQSANLLAEAKKLNDAQAPKC

SEQ ID NO: 213 - protein, 59 aa, synthetic construct; identical to SEQ ID NO: 197

SEQ ID NO: 214 - protein, 59 aa, synthetic construct
AENKFNKERSHAAQEIDALPNLNRVQWNAFIKSLVDDPSQSANLLAEAKKLNDAQAPKC

SEQ ID NO: 215 - protein, 59 aa, synthetic construct
AENKFNKELIAAAKEIDELPNLNRRQWNAFIKSLVDDPSQSANLLAEAKKLNDAQAPKC

SEQ ID NO: 216 - protein, 59 aa, synthetic construct
AENKFNKERIRAAEEIDGLPNLNRWQWNAFIKSLVDDPSQSANLLAEAKKLNDAQAPKC

SEQ ID NO: 217 - protein, 59 aa, synthetic construct
AENKFNKELVQAAREIDALPNLNRQQWNAFIKSLVDDPSQSANLLAEAKKLNDAQAPKC

SEQ ID NO: 218 - protein, 59 aa, synthetic construct
AENKFNKERREAAREIDNLPNLNRRQWNAFIKSLVDDPSQSANLLAEAKKLNDAQAPKC

SEQ ID NO: 219 - protein, 59 aa, synthetic construct
AENKFNKELRWAAGEIDKLPNLNRKQWNAFIKSLVDDPSQSANLLAEAKKLNDAQAPKC

SEQ ID NO: 220 - protein, 59 aa, synthetic construct
AENKFNKELSRAAEEIDRLPNLNRVQWNAFIKSLVDDPSQSANLLAEAKKLNDAQAPKC

SEQ ID NO: 221 - protein, 59 aa, synthetic construct
AENKFNKELIEAAREIDELPNLNRRQWNAFIKSLVDDPSQSANLLAEAKKLNDAQAPKC

SEQ ID NO: 222 - protein, 59 aa, synthetic construct
AENKFNKELIAAAKEIDDLPNLNREQWNAFIKSLVDDPSQSANLLAEAKKLNDAQAPKC

SEQ ID NO: 223 - protein, 59 aa, synthetic construct
AENKFNKERVRAAEEIDSLPNLNRQQWNAFIKSLVDDPSQSANLLAEAKKLNDAQAPKC

SEQ ID NO: 224 - protein, 59 aa, synthetic construct
AENKFNKELIDAAAEIDKLPNLNRGQWNAFIKSLVDDPSQSANLLAEAKKLNDAQAPKC

SEQ ID NO: 225 - protein, 59 aa, synthetic construct
AENKFNKELIDAAAEIDKLPNLNRSQWNAFIKSLVDDPSQSANLLAEAKKLNDAQAPKC

SEQ ID NO: 226 - protein, 59 aa, synthetic construct
AENKFNKELIEAAEEIDRLPNLNRQQWNAFIKSLVDDPSQSANLLAEAKKLNDAQAPKC

SEQ ID NO: 227 - protein, 59 aa, synthetic construct
AENKFNKELIEAAREIDELPNLNRKQWNAFIKSLVDDPSQSANLLAEAKKLNDAQAPKC

SEQ ID NO: 228 - protein, 59 aa, synthetic construct
AENKFNKELREAAEEIDSLPNLNRKQWNAFIKSLVDDPSQSANLLAEAKKLNDAQAPKC

SEQ ID NO: 229 - protein, 59 aa, synthetic construct
AENKFNKELVDAAREIDDLPNLNRGQWNAFIKSLVDDPSQSANLLAEAKKLNDAQAPKC

SEQ ID NO: 230 - protein, 59 aa, synthetic construct
AENKFNKELREAAAEIDALPNLNRKQWNAFIKSLVDDPSQSANLLAEAKKLNDAQAPKC

SEQ ID NO: 231 - protein, 59 aa, synthetic construct
AENKFNKELIEAAAEIDKLPNLNRAQWNAFIKSLVDDPSQSANLLAEAKKLNDAQAPKC

SEQ ID NO: 232 - protein, 59 aa, synthetic construct
AENKFNKELREAAGEIDRLPNLNRRQWNAFIKSLVDDPSQSANLLAEAKKLNDAQAPKC

SEQ ID NO: 233 - protein, 59 aa, synthetic construct
AENKFNKELVRAAEEIDALPNLNRKQWNAFIKSLVDDPSQSANLLAEAKKLNDAQAPKC

SEQ ID NO: 234 - protein, 59 aa, synthetic construct
AENKFNKERSRAAAEIDALPNLNRRQWNAFIKSLVDDPSQSANLLAEAKKLNDAQAPKC

SEQ ID NO: 235 - protein, 59 aa, synthetic construct
AENKFNKELIRAASEIDKLPNLNRRQWNAFIKSLRDDPSQSANLLAEAKKLNDAQAPKC

SEQ ID NO: 236 - protein, 59 aa, synthetic construct
AENKFNKELIKAAQEIDRLPNLNRTQWNAFIRSLVDDPSQSANLLAEAKKLNDAQAPKC

SEQ ID NO: 237 - protein, 59 aa, synthetic construct
AENKFNKELIEAAAEIDALPNLNRRQWNAFIKSLVDDPSQSANLLAEAKKLNDAQAPKC

SEQ ID NO: 238 - protein, 59 aa, synthetic construct
AENKFNKELIWAAGEIDKLPNLNRRQWNAFIKSLVDDPSQSANLLAEAKKLNDAQAPKC

SEQ ID NO: 239 - protein, 59 aa, synthetic construct
AENKFNKERLAAAEEIDNLPNLNRRQWNAFIKSLVDDPSQSANLLAEAKKLNDAQAPKC

SEQ ID NO: 240 - protein, 59 aa, synthetic construct
AENKFNKELRKAAEEIDALPNLNRKQWNAFIKSLVDDPSQSANLLAEAKKLNDAQAPKC

SEQ ID NO: 241 - protein, 59 aa, synthetic construct
AENKFNKELIAAAREIDSLPNLNRRQWNAFIKSLVDDPSQSANLLAEAKKLNDAQAPKC

SEQ ID NO: 242 - protein, 59 aa, synthetic construct
AENKFNKERVKAAEEIDKLPNLNRRQWNAFIKSLVDDPSQSANLLAEAKKLNDAQAPKC

SEQ ID NO: 243 - protein, 59 aa, synthetic construct
AENKFNKELIHAAEEIDRLPNLNRNQWNAFIKSLVDDPSQSANLLAEAKKLNDAQAPKC

SEQ ID NO: 244 - protein, 59 aa, synthetic construct
AENKFNKELINAAGEIDKLPNLNRKQWNAFIKSLVDDPSQSANLLAEAKKLNDAQAPKC

SEQ ID NO: 245 - protein, 59 aa, synthetic construct
AENKFNKELIEAAAEIDALPNLNRAQWNAFIKSLVDDPSQSANLLAEAKKLNDAQAPKC

SEQ ID NO: 246 - protein, 59 aa, synthetic construct; identical to SEQ ID NO: 241

SEQ ID NO: 247 - protein, 59 aa, synthetic construct
AENKFNKERVDAAREIDNLPNLNREQWNAFIRSLVDDPSQSANLLAEAKKLNDAQAPKC

SEQ ID NO: 248 - protein, 59 aa, synthetic construct
AENKFNKELRDAAAEIDRLPNLNRKQWNAFIKSLVDDPSQSANLLAEAKKLNDAQAPKC

SEQ ID NO: 249 - protein, 59 aa, synthetic construct
AENKFNKELIAAAAEIDRLPNLNRVQWNAFIKSLVDDPSQSANLLAEAKKLNDAQAPKC

SEQ ID NO: 250 - protein, 59 aa, synthetic construct
AENKFNKELRAAAEEIDKLPNLNRRQWNAFIKSLIDDPSQSANLLAEAKKLNDAQAPKC

SEQ ID NO: 251 - protein, 59 aa, synthetic construct
AENKFNKELIWAAAEIDRLPNLNREQWNAFIRSLVDDPSQSANLLAEAKKLNDAQAPKC

SEQ ID NO: 252 - protein, 59 aa, synthetic construct
AENKFNKELRAAAKEIDNLPNLNRRQWNAFIKSLVDDPSQSANLLAEAKKLNDAQAPKC

SEQ ID NO: 253 - protein, 59 aa, synthetic construct
AENKFNKELVQAAKEIDNLPNLNRSQWNAFIRSLVDDPSQSANLLAEAKKLNDAQAPKC

SEQ ID NO: 254 - protein, 59 aa, synthetic construct
AENKFNKELIEAAGEIDALPNLNRRQWNAFIKSLVDDPSQSANLLAEAKKLNDAQAPKC

SEQ ID NO: 255 - protein, 59 aa, synthetic construct
AENKFNKELVKAAAEIDALPNLNRRQWNAFIKSLVDDPSQSANLLAEAKKLNDAQAPKC

SEQ ID NO: 256 - protein, 59 aa, synthetic construct
AENKFNKELREAAREIDSLPNLNRSQWNAFIKSLVDDPSQSANLLAEAKKLNDAQAPKC

SEQ ID NO: 257 - protein, 59 aa, synthetic construct
AENKFNKELLEAAKEIDELPNLNRRQWNAFIKSLVDDPSQSANLLAEAKKLNDAQAPKC

SEQ ID NO: 258 - protein, 59 aa, synthetic construct
AENKFNKELVEAAREIDELPNLNRSQWNAFIKSLVDDPSQSANLLAEAKKLNDAQAPKC

SEQ ID NO: 259 - protein, 59 aa, synthetic construct
AENKFNKELREAAAEIDALPNLNRSQWNAFIKSLVDDPSQSANLLAEAKKLNDAQAPKC

SEQ ID NO: 260 - protein, 59 aa, synthetic construct
AENKFNKELVRAAEEIDRLPNLNRAQWNAFIKSLVDDPSQSANLLAEAKKLNDAQAPKC

SEQ ID NO: 261 - protein, 59 aa, synthetic construct
AENKFNKELRAAAAEIDSLPNLNRGQWNAFIKSLVDDPSQSANLLAEAKKLNDAQAPKC

SEQ ID NO: 262 - protein, 59 aa, synthetic construct
AENKFNKELIRAAREIDELPNLNRMQWNAFIKSLVDDPSQSANLLAEAKKLNDAQAPKC

SEQ ID NO: 263 - protein, 59 aa, synthetic construct
AENKFNKELRDAAREIDSLPNLNRRQWNAFIRSLVDDPSQSANLLAEAKKLNDAQAPKC

SEQ ID NO: 264 - protein, 59 aa, synthetic construct
AENKFNKELREAAREIDELPNLNRRQWNAFIKSLVDDPSQSANLLAEAKKLNDAQAPKC

SEQ ID NO: 265 - protein, 59 aa, synthetic construct
AENKFNKELIRAAEEIDKLPNLNRKQWNAFIKSLVDDPSQSANLLAEAKKLNDAQAPKC

SEQ ID NO: 266 - protein, 59 aa, synthetic construct
AENKFNKELLKAAREIDELPNLNRRQWNAFIRSLVDDPSQSANLLAEAKKLNDAQAPKC

SEQ ID NO: 267 - protein, 59 aa, synthetic construct
AENKFNKELIAAAREIDGLPNLNRKQWNAFIKSLVDDPSQSANLLAEAKKLNDAQAPKC

SEQ ID NO: 268 - protein, 59 aa, synthetic construct
AENKFNKELVAAAAEIDGLPNLNRKQWNAFIHSLVDDPSQSANLLAEAKKLNDAQAPKC

SEQ ID NO: 269 - protein, 59 aa, synthetic construct
AENKFNKELRDAAREIDALPNLNRAQWNAFIKSLVDDPSQSANLLAEAKKLNDAQAPKC

SEQ ID NO: 270 - protein, 59 aa, synthetic construct
AENKFNKERINAAKEIDSLPNLNRRQWNAFIKSLVDDPSQSANLLAEAKKLNDAQAPKC

SEQ ID NO: 271 - protein, 59 aa, synthetic construct
AENKFNKELRQAAAEIDALPNLNRQQWNAFIKSLVDDPSQSANLLAEAKKLNDAQAPKC

SEQ ID NO: 272 - protein, 59 aa, synthetic construct
AENKFNKELVEAAAEIDRLPNLNRGQWNAFIKSLVDDPSQSANLLAEAKKLNDAQAPKC

SEQ ID NO: 273 - protein, 59 aa, synthetic construct
AENKFNKELREAAAEIDRLPNLNRKQWNAFIKSLVDDPSQSANLLAEAKKLNDAQAPKC

SEQ ID NO: 274 - protein, 59 aa, synthetic construct
AENKFNKELIQAAKEIDSLPNLNRRQWNAFIKSLVDDPSQSANLLAEAKKLNDAQAPKC

SEQ ID NO: 275 - protein, 59 aa, synthetic construct
AENKFNKERSMAAKEIDSLPNLNRRQWNAFIKSLVDDPSQSANLLAEAKKLNDAQAPKC

SEQ ID NO: 276 - protein, 59 aa, synthetic construct
AENKFNKELVAAAREIDDLPNLNRKQWNAFIRSLVDDPSQSANLLAEAKKLNDAQAPKC

SEQ ID NO: 277 - protein, 59 aa, synthetic construct
AENKFNKELVRAAEEIDRLPNLNRLQWNAFIKSLIDDPSQSANLLAEAKKLNDAQAPKC

SEQ ID NO: 278 - protein, 59 aa, synthetic construct
AENKFNKELREAAAEIDDLPNLNRAQWNAFIKSLVDDPSQSANLLAEAKKLNDAQAPKC

SEQ ID NO: 279 - protein, 59 aa, synthetic construct
AENKFNKELVEAAKEIDSLPNLNRRQWNAFIKSLVDDPSQSANLLAEAKKLNDAQAPKC

SEQ ID NO: 280 - protein, 59 aa, synthetic construct
AENKFNKELINAAGEIDRLPNLNRRQWNAFIKSLVDDPSQSANLLAEAKKLNDAQAPKC

SEQ ID NO: 281 - protein, 59 aa, synthetic construct
AENKFNKELLSAAAEIDSLPNLNRKQWNAFIKSLVDDPSQSANLLAEAKKLNDAQAPKC

SEQ ID NO: 282 - protein, 59 aa, synthetic construct
AENKFNKELRDAAAEIDKLPNLNRRQWNAFIKSLVDDPSQSANLLAEAKKLNDAQAPKC

SEQ ID NO: 283 - protein, 59 aa, synthetic construct
AENKFNKELRQAAAEIDKLPNLNRSQWNAFIQSLVDDPSQSANLLAEAKKLNDAQAPKC

SEQ ID NO: 284 - protein, 59 aa, synthetic construct
AENKFNKELREAAAEIDALPNLNRVQWNAFIKSLRDDPSQSANLLAEAKKLNDAQAPKC

SEQ ID NO: 285 - protein, 59 aa, synthetic construct
AENKFNKELVAAAEEIDRLPNLNRAQWNAFIKSLVDDPSQSANLLAEAKKLNDAQAPKC

SEQ ID NO: 286 - protein, 59 aa, synthetic construct
AENKFNKELIDAAAEIDKLPNLNRNQWNAFIKSLVDDPSQSANLLAEAKKLNDAQAPKC

SEQ ID NO: 287 - protein, 59 aa, synthetic construct
AENKFNKERINAAAEIDGLPNLNRQQWNAFIKSLVDDPSQSANLLAEAKKLNDAQAPKC

SEQ ID NO: 288 - protein, 59 aa, synthetic construct
AENKFNKELVRAAEEIDNLPNLNRKQWNAFIKSLVDDPSQSANLLAEAKKLNDAQAPKC

SEQ ID NO: 289 - protein, 59 aa, synthetic construct
AENKFNKELIRAAKEIDELPNLNRRQWNAFIRSLVDDPSQSANLLAEAKKLNDAQAPKC

SEQ ID NO: 290 - protein, 59 aa, synthetic construct
AENKFNKELIQAAREIDSLPNLNRRQWNAFIKSLVDDPSQSANLLAEAKKLNDAQAPKC

SEQ ID NO: 291 - protein, 59 aa, synthetic construct
AENKFNKELIDAAREIDDLPNLNRQQWNAFIKSLLDDPSQSANLLAEAKKLNDAQAPKC

SEQ ID NO: 292 - protein, 59 aa, synthetic construct
AENKFNKELLKAADEIDALPNLNRAQWNAFIKSLVDDPSQSANLLAEAKKLNDAQAPKC

SEQ ID NO: 293 - protein, 59 aa, synthetic construct
AENKFNKELLEAAAEIDSLPNLNRRQWNAFIKSLVDDPSQSANLLAEAKKLNDAQAPKC

SEQ ID NO: 294 - protein, 59 aa, synthetic construct
AENKFNKELVDAAREIDTLPNLNRRQWNAFIKSLVDDPSQSANLLAEAKKLNDAQAPKC

SEQ ID NO: 295 - protein, 59 aa, synthetic construct
AENKFNKELIEAAREIDKLPNLNRRQWNAFIRSLVDDPSQSANLLAEAKKLNDAQAPKC

SEQ ID NO: 296 - protein, 59 aa, synthetic construct
AENKFNKELVAAAREIDNLPNLNRRQWNAFIKSLVDDPSQSANLLAEAKKLNDAQAPKC

SEQ ID NO: 297 - protein, 59 aa, synthetic construct
AENKFNKELVEAAAEIDGLPNLNRDQWNAFIKSLRDDPSQSANLLAEAKKLNDAQAPKC

SEQ ID NO: 298 - protein, 59 aa, synthetic construct
AENKFNKELRNAAKEIDGLPNLNRRQWNAFIKSLVDDPSQSANLLAEAKKLNDAQAPKC

SEQ ID NO: 299 - protein, 59 aa, synthetic construct
AENKFNKELRHAAREIDGLPNLNRTQWNAFIKSLVDDPSQSANLLAEAKKLNDAQAPKC

SEQ ID NO: 300 - protein, 59 aa, synthetic construct
AENKFNKELVEAAREIDTLPNLNRRQWNAFIKSLRDDPSQSANLLAEAKKLNDAQAPKC

SEQ ID NO: 301 - protein, 59 aa, synthetic construct
AENKFNKELVAAAAEIDALPNLNRRQWNAFIKSLVDDPSQSANLLAEAKKLNDAQAPKC

SEQ ID NO: 302 - protein, 59 aa, synthetic construct
AENKFNKELRTAAKEIDDLPNLNRKQWNAFIKSLVDDPSQSANLLAEAKKLNDAQAPKC

SEQ ID NO: 303 - protein, 59 aa, synthetic construct
AENKFNKELLRAAREIDSLPNLNRTQWNAFIKSLVDDPSQSANLLAEAKKLNDAQAPKC

SEQ ID NO: 304 - protein, 59 aa, synthetic construct
AENKFNKERIRAAREIDALPNLNRQQWNAFIKSLVDDPSQSANLLAEAKKLNDAQAPKC

SEQ ID NO: 305 - protein, 59 aa, synthetic construct
AENKFNKELRAAAEEIDRLPNLNRRQWNAFIKSLVDDPSQSANLLAEAKKLNDAQAPKC

SEQ ID NO: 306 - protein, 59 aa, synthetic construct
AENKFNKELIEAAAEIDALPNLNRKQWNAFISSLVDDPSQSANLLAEAKKLNDAQAPKC

SEQ ID NO: 307 - protein, 59 aa, synthetic construct
AENKFNKERIEAAREIDELPNLNRKQWNAFISSLVDDPSQSANLLAEAKKLNDAQAPKC

SEQ ID NO: 308 - protein, 59 aa, synthetic construct
AENKFNKELLAAAKEIDELPNLNRAQWNAFIKSLVDDPSQSANLLAEAKKLNDAQAPKC

SEQ ID NO: 309 - protein, 59 aa, synthetic construct
AENKFNKELINAAREIDELPNLNRRQWNAFIKSLVDDPSQSANLLAEAKKLNDAQAPKC

SEQ ID NO: 310 - protein, 59 aa, synthetic construct; identical to SEQ ID NO: 255

SEQ ID NO: 311 - protein, 59 aa, synthetic construct; identical to SEQ ID NO: 215

SEQ ID NO: 312 - protein, 59 aa, synthetic construct
AENKFNKELISAAREIDKLPNLNRQQWNAFIKSLVDDPSQSANLLAEAKKLNDAQAPKC

SEQ ID NO: 313 - protein, 59 aa, synthetic construct
AENKFNKELREAAEEIDKLPNLNRWQWNAFIKSLVDDPSQSANLLAEAKKLNDAQAPKC

SEQ ID NO: 314 - protein, 59 aa, synthetic construct
AENKFNKELVEAAREIDGLPNLNRAQWNAFIKSLVDDPSQSANLLAEAKKLNDAQAPKC

SEQ ID NO: 315 - protein, 59 aa, synthetic construct
AENKFNKELVAAAQEIDNLPNLNRKQWNAFISSLVDDPSQSANLLAEAKKLNDAQAPKC

SEQ ID NO: 316 - protein, 59 aa, synthetic construct
AENKFNKELRNAAAEIDKLPNLNRRQWNAFIKSLVDDPSQSANLLAEAKKLNDAQAPKC

SEQ ID NO: 317 - protein, 59 aa, synthetic construct
AENKFNKELIQAASEIDALPNLNRTQWNAFIKSLVDDPSQSANLLAEAKKLNDAQAPKC

SEQ ID NO: 318 - protein, 59 aa, synthetic construct
AENKFNKELVEAAAEIDSLPNLNRRQWNAFIKSLVDDPSQSANLLAEAKKLNDAQAPKC

SEQ ID NO: 319 - protein, 59 aa, synthetic construct
AENKFNKERIAAAQEIDALPNLNRRQWNAFIRSLVDDPSQSANLLAEAKKLNDAQAPKC

SEQ ID NO: 320 - protein, 59 aa, synthetic construct
AENKFNKELRDAAKEIDSLPNLNRRQWNAFIKSLVDDPSQSANLLAEAKKLNDAQAPKC

SEQ ID NO: 321 - protein, 59 aa, synthetic construct
AENKFNKELVAAAKEIDKLPNLNRKQWNAFIKSLVDDPSQSANLLAEAKKLNDAQAPKC

SEQ ID NO: 322 - protein, 59 aa, synthetic construct
AENKFNKELIKAAREIDELPNLNRKQWNAFIKSLIDDPSQSANLLAEAKKLNDAQAPKC

SEQ ID NO: 323 - protein, 59 aa, synthetic construct
AENKFNKELVEAASEIDGLPNLNRNQWNAFIKSLVDDPSQSANLLAEAKKLNDAQAPKC

SEQ ID NO: 324 - protein, 59 aa, synthetic construct
AENKFNKELLRAAEEIDNLPNLNRAQWNAFIKSLVDDPSQSANLLAEAKKLNDAQAPKC

SEQ ID NO: 325 - protein, 59 aa, synthetic construct
AENKFNKELIKAAKEIDALPNLNRRQWNAFIKSLVDDPSQSANLLAEAKKLNDAQAPKC

SEQ ID NO: 326 - protein, 59 aa, synthetic construct
AENKFNKELRRAAAEIDSLPNLNRDQWNAFIRSLIDDPSQSANLLAEAKKLNDAQAPKC

SEQ ID NO: 327 - protein, 59 aa, synthetic construct; identical to SEQ ID NO: 314

SEQ ID NO: 328 - protein, 59 aa, synthetic construct
AENKFNKELVEAAREIDELPNLNRRQWNAFIRSLVDDPSQSANLLAEAKKLNDAQAPKC

SEQ ID NO: 329 - protein, 59 aa, synthetic construct
AENKFNKELVWAAAEIDRLPNLNRCQWNAFIKSLVDDPSQSANLLAEAKKLNDAQAPKC

SEQ ID NO: 330 - protein, 59 aa, synthetic construct
AENKFNKERIRAAREIDELPNLNREQWNAFIKSLVDDPSQSANLLAEAKKLNDAQAPKC

SEQ ID NO: 331 - protein, 59 aa, synthetic construct
AENKFNKELRQAAKEIDALPNLNRRQWNAFIKSLVDDPSQSANLLAEAKKLNDAQAPKC

SEQ ID NO: 332 - protein, 59 aa, synthetic construct
AENKFNKELREAAAEIDKLPNLNRKQWNAFIRSLVDDPSQSANLLAEAKKLNDAQAPKC

SEQ ID NO: 333 - protein, 59 aa, synthetic construct
AENKFNKELRRAAEEIDKLPNLNRQQWNAFIKSLVDDPSQSANLLAEAKKLNDAQAPKC

SEQ ID NO: 334 - protein, 59 aa, synthetic construct; identical to SEQ ID NO: 217

SEQ ID NO: 335 - protein, 59 aa, synthetic construct
AENKFNKELRAAAAEIDALPNLNRRQWNAFIKSLVDDPSQSANLLAEAKKLNDAQAPKC

SEQ ID NO: 336 - protein, 59 aa, synthetic construct
AENKFNKELRAAAAEIDKLPNLNRKQWNAFIKSLVDDPSQSANLLAEAKKLNDAQAPKC

SEQ ID NO: 337 - protein, 59 aa, synthetic construct
AENKFNKERIQAAKEIDELPNLNREQWNAFIKSLVDDPSQSANLLAEAKKLNDAQAPKC

SEQ ID NO: 338 - protein, 59 aa, synthetic construct
AENKFNKELVAAAEEIDKLPNLNRRQWNAFIKSLVDDPSQSANLLAEAKKLNDAQAPKC

SEQ ID NO: 339 - protein, 59 aa, synthetic construct; identical to SEQ ID NO: 288

SEQ ID NO: 340 - protein, 59 aa, synthetic construct
AENKFNKELRTAAAEIDALPNLNREQWNAFIRSLVDDPSQSANLLAEAKKLNDAQAPKC

SEQ ID NO: 341 - protein, 59 aa, synthetic construct
AENKFNKELVEAAAEIDALPNLNRKQWNAFIKSLVDDPSQSANLLAEAKKLNDAQAPKC

SEQ ID NO: 342 - protein, 59 aa, synthetic construct
AENKFNKELIKAAEEIDKLPNLNRMQWNAFIRSLVDDPSQSANLLAEAKKLNDAQAPKC

SEQ ID NO: 343 - protein, 59 aa, synthetic construct
AENKFNKELREAAAEIDNLPNLNRRQWNAFIKSLIDDPSQSANLLAEAKKLNDAQAPKC

SEQ ID NO: 344 - protein, 59 aa, synthetic construct
AENKFNKELIEAAREIDALPNLNRKQWNAFIRSLVDDPSQSANLLAEAKKLNDAQAPKC

SEQ ID NO: 345 - protein, 59 aa, synthetic construct
AENKFNKELREAAQEIDSLPNLNRQQWNAFIKSLVDDPSQSANLLAEAKKLNDAQAPKC

SEQ ID NO: 346 - protein, 59 aa, synthetic construct
AENKFNKELRSAAEEIDSLPNLNRRQWNAFIKSLVDDPSQSANLLAEAKKLNDAQAPKC

SEQ ID NO: 347 - protein, 59 aa, synthetic construct
AENKFNKELVEAAAEIDNLPNLNRMQWNAFIKSLVDDPSQSANLLAEAKKLNDAQAPKC

SEQ ID NO: 348 - protein, 59 aa, synthetic construct
AENKFNKELVEAAGEIDNLPNLNRRQWNAFIRSLVDDPSQSANLLAEAKKLNDAQAPKC

SEQ ID NO: 349 - protein, 59 aa, synthetic construct
AENKFNKELSKAAAEIDALPNLNRRQWNAFIKSLVDDPSQSANLLAEAKKLNDAQAPKC

SEQ ID NO: 350 - protein, 59 aa, synthetic construct
AENKFNKELLWAAGEIDRLPNLNRQQWNAFIKSLVDDPSQSANLLAEAKKLNDAQAPKC

SEQ ID NO: 351 - protein, 59 aa, synthetic construct
AENKFNKELRQAAAEIDGLPNLNRRQWNAFIKSLVDDPSQSANLLAEAKKLNDAQAPKC

SEQ ID NO: 352 - protein, 498 aa, Homo sapiens
VVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFK

SEQ ID NO: 353 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

(19) United States
(12) Patent Application Publication
(10) Pub. No.: US 2024/0197930 A1
(43) Pub. Date: Jun. 20, 2024

(54) PET TRACER

(71) Applicant: Antaros Tracer AB, Mölndal (SE)

(72) Inventor: Olof ERIKSSON, Uppsala (SE)

(21) Appl. No.: 18/529,406

(22) Filed: Dec. 5, 2023

(30) Foreign Application Priority Data
Dec. 14, 2022 (SE) 2251463-2

(51) Int. Cl. A61K 51/08

(52) U.S. Cl. CPC A61K 51/088

(57) ABSTRACT

The invention provides conjugated binding molecules comprising platelet-derived growth factor receptor beta (PDGF-Rβ) binding polypeptides conjugated to radionuclides chelated by a Restrained Complexing Agent (RESCA) chelator, wherein Aluminum-Fluorine-18 is the preferred radionuclide. The PDGF-Rβ binding polypeptide comprises a platelet derived growth factor receptor beta binding motif, PBM, which motif consists of an amino acid sequence as defined herein, wherein the PDGF-Rβ-binding polypeptide binds to PDGF-Rβ such that the K_{D} value of the interaction is at most 1×10⁻⁶ M. Also provided are methods and uses of said conjugated binding molecule in imaging and diagnosis of PDGF-Rβ-related conditions, such as fibrosis.

### SEQUENCE LISTING

The Sequence Listing submitted herewith, entitled “Dec-5-2023-sequence-listing.xml”, created Nov. 14, 2023 and having a size of 339,438 bytes, is incorporated herein by reference.

### TECHNICAL FIELD

The present disclosure relates conjugated binding molecules that bind to the extracellular domain of platelet-derived growth factor receptor beta (PDGF-Rβ). More specifically, the conjugated binding molecules comprise a PDGF-Rβ binding polypeptide linked to a chelator comprising a radionuclide, wherein the chelator is Restrained Complexing Agent (RESCA). The conjugated binding molecules may be used in diagnosis or medical imaging, such as for evaluating ongoing rate of fibrogenesis and resulting fibrosis.

### BACKGROUND

Development of fibrous connective tissue as a reparative response to injury or damage is referred to as fibrosis. Fibrosis may both refer to the connective tissue deposition that occurs as part of normal healing, and to the excess tissue deposition that occurs as a pathological process. Some of the main types of fibrosis occurring in the body is pulmonary fibrosis, cardiac fibrosis and hepatic (liver) fibrosis. When scar tissue builds up and takes over most of the liver, this is a more serious problem called hepatic cirrhosis. Cirrhosis refers to the scar tissue and nodules that replace liver tissue and disrupt liver function. The condition is usually caused by alcoholism, fatty liver disease, hepatitis B or hepatitis C. Patients with fatty liver disease exhibit excess fat deposits in the liver, seen both in patients who are alcoholics as well as in patients who drink little to no alcohol. This non-alcohol induced fatty liver disease is referred to as non-alcoholic fatty liver disease (NAFLD), and is also known as simple steatosis. If NAFLD progresses it may lead to more severe stages such as cirrhosis and non-alcoholic steatohepatitis (NASH).

Fibrosis is an excess amount of collagen or extracellular matrix (ECM) in tissue. Fibrosis is defined by the overgrowth, hardening, and/or scarring of various tissues and is attributed to excess deposition of extracellular matrix components, including collagen, which can be detrimental to health and lead to organ failure. It is a common pathological response to tissue insults such as hyperglycemia, dyslipidemia and hypertension, hence often present in patients with diabetes. In long-standing diabetes, structural and functional defects in the vasculature may lead to diabetes complications including retinopathy, nephropathy, myocardiopathy, and atherosclerosis. Pathological fibrosis can affect many different tissues, including but not limited to liver, lung, intestine, heart, kidney, brain and pancreas. Fibrosis is also associated to many types of tumors.

The amount of collagen in tissue is dependent on the balance between fibrogenesis (deposition of new collagen and ECM) and fibrolysis (breakdown of collagen). The cell types commonly responsible for fibrogenesis are fibroblasts and mesenchymal stromal cells (MSCs). Here on we will refer to collagen and ECM producing cells as fibrogenic cells or fibroblasts.

Assessment of fibrosis degree or active fibrogenesis is today performed by invasive biopsy of tissue. This makes it challenging to diagnose and stage fibrosis, select optimal treatment for individual patients and to evaluate the effect of novel anti-fibrotic interventions. Accordingly, there is a need for new and enhanced methods for detecting, diagnosing and assessing fibrosis, degree of fibrosis and/or active fibrogenesis.

### SUMMARY

An object of the present disclosure is to provide novel and enhanced conjugated binding molecule which may be used in diagnosis and medical imaging. This object is obtained by a conjugated binding molecule that specifically binds platelet derived growth factor receptor beta (PDGF-Rβ), the conjugated binding molecule comprising i) at least one binding polypeptide, the binding polypeptide being platelet derived growth factor receptor beta (PDGF-Rβ) binding polypeptide and ii) at least one agent, the at least one agent being indirectly joined to the binding polypeptide via a linker in the form of a chelator, wherein the at least one agent comprises or consists of a radionuclide and wherein the chelator is Restrained Complexing Agent (RESCA). The radionuclide is typically a detectable agent, and in a preferred embodiment the radionuclide is Fluorine-18 conjugated to aluminum, i.e. Aluminum-Fluorine-18 (Al¹⁸F).

According to one aspect of the invention, the at least one PDGF-Rβ binding polypeptide comprises a platelet derived growth factor receptor beta binding motif, PBM, which motif consists of the amino acid sequence selected from

i)
(SEQ ID NO: 1)
EX₂X₃X₄AAX₇EIDX₁₁LPNLX₁₆X₁₇X₁₈QWNAFIX₂₅X₂₆LX₂₈X₂₉,

wherein, independently of each other,
X₂ is selected from L, R and I;
X₃ is selected from R, I, L, V, K, Q, S, H, and A;
X₄ is selected from A, R, N, D, Q, E, H, K, M, S, T, W, F and V;
X₇ is selected from A, R, D, Q, E, G, K and S;
X₁₁ is selected from A, R, N, D, E, G, K, S, T and Q;
X₁₆ is selected from N and T;
X₁₇ is selected from R and K;
X₁₈ is selected from A, R, N, D, C, Q, E, G, L, K, M, S, T, W and V;
X₂₅ is selected from K, R, Q, H, S, G and A;
X₂₆ is selected from S and K;
X₂₈ is selected from V, R, I, L and A;
X₂₉ is selected from D and K;
and
ii) an amino acid sequence which has at least 89% identity to the sequence defined in i), and wherein the PDGF-Rβ-binding polypeptide binds to PDGF-R3 such that the K_{D} value of the interaction is at most 1×10⁻⁶ M.

According to a preferred embodiment, the PDGF-Rβ-binding motif forms part of a three-helix bundle protein domain, wherein in which said PDGF-Rβ-binding motif essentially forms part of two alpha helices with an interconnecting loop, within said three-helix bundle protein domain.

According to some embodiments, at least one PDGF-R3 binding polypeptide comprises an amino acid sequence selected from: VDNKFNK-[PBM]-DPSQSANLLAEAKKLNDAQAPK (SEQ ID NO:2); and AENKFNK-[PBM]-DPSQSANLLAEAKKLNDAQAPKC (SEQ ID NO:3), wherein [PBM] is an PDGF-Rβ-binding motif as defined above. In a further embodiment, at least one PDGF-R3 binding polypeptide comprises an amino acid sequence selected from SEQ ID NO: 4-351. In yet a further embodiment, at least one PDGF-R3 binding polypeptide is a so called Z-molecule, such as in particular a molecule comprising the amino acid sequence: AENKFNKELIEAAAEIDALPNLNRRQWNAFIKSLVDDPSQSANLLAEAKKLNDAQAPKC (SEQ ID NO: 237). In one embodiment, the conjugated binding molecule having the construct: A1¹³F-RESCA-Z.

In some aspects is provided conjugated binding molecules, which may be used in Position Emission Tomography (PET) imaging.

In yet another aspect is provided conjugated binding molecules, which may be used in the assessment of fibrosis, fibrosis degree and/or active fibrogenesis.

In yet another aspect is provided an in vivo method for detecting activated fibroblast cells, and/or activated mesenchymal stromal cells, in a subject by detecting expression of platelet derived growth factor receptor beta (PDGF-Rβ), the method comprising (i) administering one or more conjugated binding molecule as defined above to the subject; and (ii) detecting that the one or more conjugated binding molecule has bound fibroblast cells, and/or mesenchymal stromal cells, wherein the bound fibroblast cells and/or bound mesenchymal stromal cells are determined as being activated.

### BRIEF DESCRIPTION OF THE DRAWINGS

The above, as well as additional objects, features and advantages of the present inventive concept, will be better understood through the following illustrative and non-limiting detailed description of different embodiments of the present inventive concept, with reference to the appended drawings, wherein:

FIG. 1 illustrates the generic concept of a binding molecule conjugated to an agent as payload.

FIG. 2 illustrates a conjugated binding molecule of the present disclosure which comprise one PDGF-Rβ binding polypeptide (probe) and at least one agent, where the at least one agent is being indirectly joined to the binding polypeptide via a linker in the form of a chelator.

FIG. 3 illustrates conjugated binding molecule of the present disclosure interacting with a target cell-surface receptor, wherein the conjugated binding molecule comprise one PDGF-Rβ binding polypeptide (targeting ligand)) and at least one agent comprising a radionuclide in a chelator, where the at least one agent is being joined to the binding polypeptide via a spacer. FIG. 4 illustrates conjugated binding molecule of the present disclosure interacting with a PDGF-Rβ target cell-surface receptor, wherein the conjugated binding molecule comprise one PDGF-RB binding polypeptide (targeting ligand) and at least one agent comprising a Fluorine-18 radionuclide in a RESCA chelator, where the at least one agent is being joined to the binding polypeptide via a linker, with no spacer.

FIG. 5 illustrates the structure of the RESCA chelator with a malemide group as used in the present conjugated binding molecules.

FIG. 6 shows RESCA chelator conjugated to a polypeptide via malemide chemistry.

FIG. 7 shows AI[18F] coordinated in a RESCA chelator, conjugated to a polypeptide via malemide chemistry.

FIG. 8 shows in vitro autoradiography on fibrotic human liver biopsy.

FIG. 9 shows PET-MRI scans on U-87 (PDGFRB expressing cells) xenograft mice.

FIG. 10 shows ex vivo biodistribution on U-87 (PDGFRB expressing cells) xenograft mice.

FIG. 11 shows ex vivo biodistribution on U-87 xenograft mice in a comparative biodistribution with other analogues, showing % ID/g uptake.

FIG. 12 shows ex vivo biodistribution on U-87 xenograft mice in a comparative biodistribution with other analogues, showing tumor-to-tissue uptake.

FIGS. 13A-13D show ex vivo biodistribution on U-87 xenograft mice in a comparative biodistribution with other analogues, showing individual graphs of % ID/g uptake.

FIG. 14 shows PET scan in a liver fibrosis mouse model.

FIG. 15 shows ex vivo biodistribution in a liver fibrosis mouse model.

FIG. 16 shows ex vivo autoradiography in a liver fibrosis mouse model.

The figures are not necessarily to scale, and generally only show parts that are necessary in order to elucidate the inventive concept, wherein other parts may be omitted or merely suggested.

### DETAILED DESCRIPTION

The present disclosure relates to new conjugated binding molecules, which selectively bind platelet-derived growth factor receptor beta (PDGF-Rβ), and which carry radionuclides via chelators. The conjugated binding molecules may be used in imaging applications, for in vitro and in vivo diagnosis. Thus, an aim of the present disclosure is to provide new and enhanced conjugated binding molecules specific for PDGF-Rβ, which may be used in diagnosis and medical imaging of fibrosis and fibrogenesis.

Today, assessment of fibrosis degree or active fibrogenesis is performed by invasive biopsy of tissue, which has several drawbacks compared to non-invasive methods. Non-invasive medical imaging techniques can theoretically be used to enable biopsy-free assessment of fibrosis in patients, provided that a suitable imaging agent is available. Position Emission Tomography (PET) is a highly sensitive biomedical imaging technique using radioactively labeled contrast agents, also referred to as radiopharmaceuticals, to visualize and quantify receptors and molecules. PET is routinely used in healthcare to e.g., localize and stage tumors in the oncological setting. A radioactive tracer is a chemical compound in which one or more atoms have been replaced by a radioisotope. As radiopharmaceuticals may also refer to therapeutic substances, the term radioactive tracer or “radiotracer” may be used for imaging applications.

The platelet-derived growth factor receptor beta (PDGF-Rβ) is a membrane-spanning tyrosine kinase. Human PDGF-Rβ has UniProt number P09619. The ligand PDGF is composed of combinations of the homologous chains A, B, C and D, combined to either homo- or heterodimers. PDGF-Rβ binds PDGF-BB with high affinity and PDGF-AB with lower affinity.

Ligand binding leads to dimerization and trans-phosphorylation of tyrosines in the intracellular kinase domain of the receptors. PDGF is an important factor for regulating cell proliferation, cellular differentiation, cell growth and development. PDGF-Rβ is implicated in angiogenesis and in early stages of fibrosis. This receptor represents an attractive and potentially valuable target, e.g., for treatment and molecular imaging in for example oncologic and fibrotic diseases.

Activated fibroblasts express the surface antigen PDGF-Rβ, while it is absent from resting fibroblasts. PDGF-Rβ is therefore a putative target for PET tracers for imaging of fibrogenesis. Molecules that bind to the extracellular domain of PDGF-Rβ has previously been described in a patent application published as WO2009/077175, which is incorporated by reference herein to the extent allowed.

In V. Tolmachev et al., J Nucl Med 2014; 55:294-300 (incorporated by reference herein to the extent allowed), it was studied if radiolabeled derivatives of the molecule Z02465 from WO2009/077175 would be suitable for radionuclide imaging of PDGF-Rβ expression in vivo. Z02465 was redesigned, a unique thiol group was created by introducing a cysteine at the C terminus, enabling site-specific labeling using thiol-directed chemistry, and the first 2 amino acids at the N terminus were changed from VD- to AE-, because such substitution improves biodistribution of the molecules. The resulting variant of denoted Z09591 was labeled with ¹¹¹In and evaluated for targeting of PDGF-Rβ-expressing U-87 MG cells in vitro and in vivo.

PET imaging of fibrosis can potentially be achieved by development of new radiopharmaceuticals binding to protein markers on the membrane surface of activated fibroblasts, e.g., the cells responsible for fibrogenesis. Such a PET radiopharmaceutical would generate images of tissue, where the achieved contrast would be proportional to the fibroblast activity, and thus the ongoing rate of fibrogenesis. Thus, the contrast of the PET pharmaceutical in tissue is crucial. For optimal contrast, the radiopharmaceutical must have both strong binding (high binding affinity) to its target, high specificity for the target, as well as low background signal in tissue and circulation. Further, good stability in vivo, low immunogenicity and toxicity are vital. Suitable pharmacokinetics include high plasma clearance and low plasma protein binding, low nonspecific uptake in nontarget tissues and kidney excretion, while hepatobiliary excretion would be undesirable. Well-established bioconjugation and radiolabeling strategies are also important.

Hence, to enable PET imaging of fibrogenesis and fibrosis, it was envisaged that the PDGF-Rβ binding molecules of WO2009/077175 (and modified variants thereof) could be used as the binding part of conjugated binding molecules to be used as radiopharmaceuticals in PET imaging. Thus, the PDGF-Rβ binding molecules of WO2009/077175 were functionalized with several different moieties, to enable radiolabeling and PET imaging of PDGF-Rβ. Surprisingly, the PDGF-Rβ binding molecules functionalized with Restrained Complexing Agent (RESCA), displayed markedly increased contrast in several tissues with PDGFRB expression, compared with other radiolabeling strategies.

Aspects of the present disclosure will be described more fully hereinafter with reference to the accompanying drawings. The conjugated binding molecules and methods disclosed herein can, however, be realized in many different forms and should not be construed as being limited to the aspects set forth herein. Like numbers in the drawings refer to like elements throughout.

The terminology used herein is for the purpose of describing particular aspects of the disclosure only, and is not intended to limit the disclosure. As used herein, the singular forms “a”, “an” and “the” are intended to include the plural forms as well, unless the context clearly indicates otherwise.

The “conjugated binding molecules” as described herein comprise i) at least one binding polypeptide, and ii) at least one agent, the at least one agent being indirectly joined to the binding polypeptide via a linker in the form of a chelator. This could also be phrased as that the at least one agent being indirectly joined to the binding polypeptide via a chelator. Thus, in one aspect the chelator may be regarded as a linker to carry the radioligand. In other aspects, the linkage between the binding molecule and the chelator/“chelator cage”, may be referred to as a linker. This could be phrased as that the at least one agent being indirectly joined to the binding polypeptide via a chelator comprising the agent, wherein the chelator is joined to the binding polypeptide via a linker. The chelator may be directly or indirectly joined to the binding polypeptide, such as by using a malemide group. In some embodiments the conjugated binding molecule comprises a binding polypeptide to which the RESCA chelator is joined via a linker, which linker may be of variable length and composition. Depending on the length, the linker may thus also function as a spacer.

The conjugated binding molecules comprise a binding polypeptide (or binding molecule), which is the part of the conjugated binding molecule which bind the target, i.e. PDGF-Rβ.

In some embodiments a non-limiting term “binding polypeptide” is used. In some aspects, “binding molecule” may be used interchangeably. The term “binding polypeptide” or “binding molecule” is used herein to denote a polypeptide/molecule that selectively binds to the extracellular domain of PDGF-Rβ, i.e. a PDGF-Rβ binding polypeptide. The PDGF-Rβ binding polypeptides of the present disclosure comprise a platelet derived growth factor receptor beta binding motif, PBM, wherein the identified PDGF-Rβ-binding motif, or “PBM”, corresponds to the target binding region of the parent scaffold, which region constitutes two alpha helices within a three-helical bundle protein domain, as defined in WO2009/077175. In the parent scaffold on which these polypeptides are based, the varied amino acid residues of the two PBM helices constitute a binding surface for interaction with the constant Fc part of antibodies. For the present binding polypeptides, the random variation of binding surface residues and the subsequent selection of variants have replaced the Fc interaction capacity with a capacity for interaction with PDGF-Rβ.

Thus, the binding polypeptides used in the conjugated binding molecules of the present disclosure is preferably selected from the molecules which comprise a platelet derived growth factor receptor beta binding motif, PBM, which motif consists of the amino acid sequence selected from

i)
(SEQ ID NO: 1)
EX₂X₃X₄AAX₇EIDX₁₁LPNLX₁₆X₁₇X₁₈QWNAFIX₂₅X₂₆LX₂₈X₂₉,

wherein, independently of each other,
X₂ is selected from L, R and I;
X₃ is selected from R, I, L, V, K, Q, S, H, and A;
X₄ is selected from A, R, N, D, Q, E, H, K, M, S, T, W, F and V;
X₇ is selected from A, R, D, Q, E, G, K and S;
X₁₁ is selected from A, R, N, D, E, G, K, S, T and Q;
X₁₆ is selected from N and T;
X₁₇ is selected from R and K;
X₁₃ is selected from A, R, N, D, C, Q, E, G, L, K, M, S, T, W and V;
X₂₅ is selected from K, R, Q, H, S, G and A;
X₂₆ is selected from S and K;
X₂₃ is selected from V, R, I, L and A;
X₂₉ is selected from D and K;
and
   ii) an amino acid sequence which has at least 89% identity to the sequence defined in i), and wherein
the PDGF-Rβ-binding polypeptide binds to PDGF-R3 such that the K_{D} value of the interaction is at most 1×10⁻⁶ M. The attained K_{D} value for the above defined binding polypeptides is demonstrated WO2009/077175.

The above definition of a class of sequence related, PDGF-Rβ-binding polypeptides is based on an analysis of a number of random polypeptide variants of a parent scaffold, that were selected for their interaction with PDGF-R3 in selection experiments. The PDGF-Rβ-binding motif forms part of a three-helix bundle protein domain. Typically, said PDGF-Rβ-binding motif essentially forms part of two alpha helices with an interconnecting loop, within said three-helix bundle protein domain. In particular embodiments of the invention, said three-helix bundle protein domain is selected from domains of bacterial receptor proteins. Non-limiting examples of such domains are the five different three-helical domains of Protein A from Staphylococcus aureus, and derivatives thereof. In some embodiments, the PBM is comprised within the protein Z derivative of domain B of staphylococcal protein A.

In some embodiments, the PDGF-R3 binding polypeptide comprises an amino acid sequence selected from:

(SEQ ID NO: 2)
VDNKFNK-[PBM]-DPSQSANLLAEAKKLNDAQAPK;
and
(SEQ ID NO: 3)
AENKFNK-[PBM]-DPSQSANLLAEAKKLNDAQAPKC,

wherein [PBM] is the PDGF-Rβ-binding motif as defined above.

In some embodiments, the PDGF-R3 binding polypeptide comprises an amino acid sequence selected from SEQ ID NO: 4-177. In other embodiments, the PDGF-R3 binding polypeptide comprises a modified sequence of the binders of SEQ ID NO: 4-177, where a cysteine has been introduced at the C terminus, enabling site-specific labeling using thiol-directed chemistry, and the first 2 amino acids at the N terminus are changed from VD- to AE-, to improve biodistribution of the molecules, these modified polypeptides comprising an amino acid sequence selected from SEQ ID NO: 178-351. In a preferred embodiment, the PDGF-R3 binding polypeptide is the Z-variant Z02465 from WO2009/077175, as defined by SEQ ID NO:63, or a modified variant Z09591, as defined by SEQ ID NO:237.

As the skilled person will realize, the function of any polypeptide, such as the PDGF-Rβ-binding capacity of the polypeptides according to the invention, is dependent on the tertiary structure of the polypeptide. It is therefore possible to make minor changes to the sequence of amino acids in a polypeptide without affecting the function thereof. Thus, the invention encompasses modified variants of the PBM of i), which are such that the resulting sequence is at least 89% identical to a sequence belonging to the class defined by i), such as at least 93% identical, such as at least 96% identical. For example, it is possible that an amino acid residue belonging to a certain functional grouping of amino acid residues (e.g., hydrophobic, hydrophilic, polar etc.) could be exchanged for another amino acid residue from the same functional group.

The skilled person will appreciate that various modifications and/or additions can be made to a binding polypeptide according to the present disclosure in order to tailor the polypeptide to a specific application. For example, a PDGF-Rβ-binding polypeptide may be extended by C terminal and/or N terminal amino acids. Said extended polypeptide is a polypeptide which has additional amino acids residues at the very first and/or the very last position in the polypeptide chain, i.e. at the N- and/or C-terminus. The polypeptide may be extended by any suitable number of additional amino acid residues, for example at least one amino acid residue. Each additional amino acid residue may individually or collectively be added in order to, for example, improve production, purification, stabilization in vivo or in vitro, coupling, or detection of the polypeptide. Such additional amino acid residues may comprise one or more amino acid residues added for the purpose of chemical coupling. One example of this is the addition of a cysteine residue. Such additional amino acid residues may also provide a “tag” for purification or detection of the polypeptide such as a His6 tag or a “myc” (c-myc) tag or a “FLAG” tag for interaction with antibodies specific to the tag. In a particular embodiment, the variant Z02465 of WO2009/077175 having the amino acid sequence VDNKFNKELIEAAAEIDALPNLNRRQWNAFIKSLVDDPSQSANLLAEAKKLNDAQAPK (SEQ ID NO:63), may be modified into Z09591 having the amino acid sequence AENKFNKELIEAAAEIDALPNLNRRQWNAFIKSLVDDPSQSANLLAEAKKLNDAQAPKC; (SEQ ID NO:237) by introducing a cysteine at the C terminus, and substituting the first 2 amino acids at the N terminus, from VD- to AE-.

The binding polypeptides described above are advantageous in that they bind well to PDGF-Rβ. The polypeptides may in particular bind to the extra-cellular domain of PDGF-Rβ. Typically, the polypeptides can be relatively short. By virtue of their small size, they are expected to exhibit a more efficient penetration in tumor and normal tissue than antibodies, as well as to have better systemic circulation properties than monoclonal antibodies (which often have too long circulation times). Thus, they are considered suitable candidates for the development of molecular imaging agents, as in the present disclosure.

As used herein, the term “capable of binding X”, wherein X is an antigen, refers to a property of a binding molecule which may be tested. More specifically, the terms “PDGF-Rβ-binding” and “binding affinity for PDGF-Rβ” as used in this specification refers to a property of a polypeptide which may be tested for example by the use of surface plasmon resonance technology, such as in a Biacore instrument (GE Healthcare). For example, PDGF-Rβ-binding affinity may be tested in an experiment in which PDGF-Rβ (e.g., human or murine PDGF-Rβ), or a fragment of PDGF-Rβ such as the extracellular domain, is immobilized on a sensor chip of the instrument, and the sample containing the polypeptide to be tested is passed over the chip, wherein the PDGF-Rβ or fragment thereof used may for example comprise the amino acid sequence represented by SEQ ID NO:352 (PDGF-Rβ extra-cellular domain) or SEQ ID NO: 353 (PDGF-Rβ). If a quantitative measure is desired, for example to determine a K_{D} value for the interaction, surface plasmon resonance methods may also be used. Binding values may for example be defined in a Biacore 2000 instrument (GE Healthcare). The skilled person is aware of said methods and others. It was demonstrated in WO2009/077175 that the preferred binding molecules of the present conjugated binding molecule have certain K_{D} values, that the PDGF-Rβ-binding polypeptides bind to PDGF-Rβ such that the K_{D} value of the interaction is at most 1×10⁻⁶ M.

The term “specificity”, sometimes referred to as “selectivity,” of the binding polypeptide for a target refers to a binding polypeptide which will bind to the target with high affinity, but typically not to other antigens. A selective or specific binding polypeptide will not, or to a low extent, cross-react with other targets than the intended antigen. Thus, by binding “specifically” it is meant that the binding polypeptide binds to its target in a manner that can be distinguished from binding to non-target molecules, more particularly that the binding molecule binds its target with greater binding affinity than with which it binds other molecules. That is, the binding molecule does not bind to other, non-target, molecules, or does not do so to an appreciable or significant degree, or binds with lower affinity to such other molecules than with which it binds PDGF-Rβ. A binding polypeptide “that specifically binds” PDGF-Rβ may alternatively be referred to as “directed against” or “that recognizes” PDGF-Rβ. In other words, PDGF-Rβ is the antigen of the binding polypeptide in the conjugated binding molecule of the present invention.

The binding polypeptide is conjugated/linked to an agent to form a conjugated binding molecule. The conjugated binding molecule of the invention may be used as an alternative to e.g., conventional antibodies conjugated drugs in various medical, veterinary and diagnostic applications. Thus, the conjugated binding molecule may be used as a therapeutic or diagnostic agent, with direct (e.g., toxic) effects on the PDGF-Rβ protein. Diagnostic applications include for example molecular imaging in order to reveal, diagnose and examine the presence of a disease, such as fibrotic diseases including pulmonary fibrosis, liver cirrhosis, scleroderma, glomerulosclerosis, tumor stroma or cardiac fibrosis, in vivo in the body of a mammalian subject. The conjugated binding molecules may be used in targeting therapeutic or diagnostic agents, both in vivo and in vitro, to cells expressing PDGF-Rβ, particularly to cells which over-express PDGF-Rβ. There is thus provided a combination of a PDGF-Rβ-binding polypeptide and a therapeutic agent in the conjugated binding molecule. Preferably, the therapeutic agent is a radionuclide. Conjugated binding molecules carrying radionuclides as the active agent, where the agent is used for diagnosis or therapy of a disease or condition, may be referred to as radiopharmaceuticals.

Molecular imaging combining imaging agents with targeting moieties in the form of a binding molecule may be used to specifically image diseased sites in the body. The binding proteins may be used in molecular imaging to target imaging agents, such as radionuclides, to the cell of interest in vivo. This gives the ability to monitor disease progression and to predict response to a specific therapeutic agent, thus enabling diagnostics and response prediction for any tissue and disease where the antigen is expressed/overexpressed. In the present case, PDGF-Rβ is overexpressed in activated fibroblast cells and/or mesenchymal stromal cells, and activation of said cells may be indicative of e.g. ongoing fibrosis. The imaging/detectable agents may be radioisotopes, and the detectable agent may be detectable by an imaging technique such as PET. In a preferred embodiment, the imaging agents are radioisotopes, i.e., Al¹⁸F, and the imaging technique is PET.

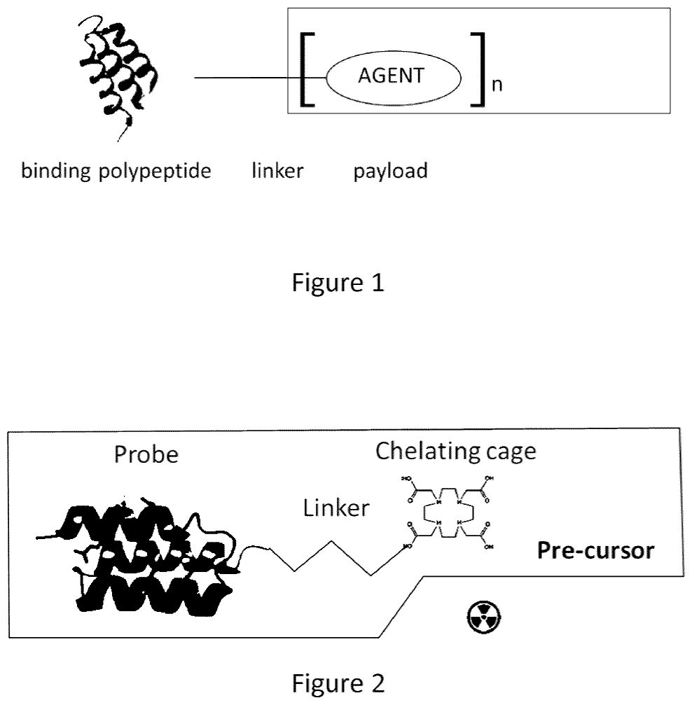

Binding polypeptides may be joined/linked to one or more agents as payload as illustrated in FIG. 1 to form conjugated binding molecules, and by recognizing and binding the target, PDGF-Rβ, they may be used to detect ongoing fibrogenesis, diagnose fibrosis and/or assess degree of fibrosis. It may also be possible to detect and quantify disease states, study distribution of drug/drug candidates in the body, and enable the measurement of efficacy of treatments. In some aspects, the agent (payload) is a detectable or imaging agent, such as a label, and the conjugated binding molecule is used in imaging. Typically, the agent is a radionuclide. The term “radionuclide” may also be referred to as “radioisotope”, and refers to a nuclide that has excess nuclear energy, making it unstable, and prone to undergo radioactive decay.

In a preferred aspect of the present disclosure, the agent may be joined to the binding polypeptide via a linker in the form of a chelator, where the agent may be coupled using a chelator (a form of indirect joining), where the chelator is joined/linked to the binding protein and chelates the agent. The conjugated binding molecules of the present disclosure comprise at least one PDGF-Rβ binding polypeptide and at least one agent, wherein the at least one agent comprises or consists of a radionuclide, where the at least one agent is being indirectly joined to the binding polypeptide via a linker in the form of a chelator, as illustrated in FIG. 2, illustrating the binding polypeptide as the “probe”, and a chelating cage for carrying the radionuclides, wherein the chelating cage is linked to the probe via a linker. In some aspects, the “linker” is in the form of malemide coupling

Chelation is a type of bonding of ions and molecules to metal ions. It involves the formation or presence of two or more separate coordinate bonds between a polydentate (multiple bonded) ligand and a single central metal atom. These ligands are called chelants, chelators, chelating agents, or sequestering agents. Chelate complexes of gadolinium are often used as contrast agents in MRI scans, and bifunctional chelate complexes of zirconium, gallium, fluorine, copper, yttrium, bromine, or iodine are often used for conjugation to monoclonal antibodies for use in antibody-based PET imaging. These chelate complexes often employ the usage of hexadentate ligands such as desferrioxamine B (DFO), and the gadolinium complexes often employ the usage of octadentate ligands such as DTPA. For radiopharmaceuticals, chelators such as derivatives of 1,4,7,10-tetraazacyclododecane-1,4,7,10, tetraacetic acid (DOTA), derivatives of deferoxamine (DFO), derivatives of diethylenetriaminepentaacetic avid (DTPA), and derivatives of S-2-(4-Isothiocyanatobenzyl)-1,4,7-triazacyclononane-1,4,7-triacetic acid (NOTA) are commonly used.

The preferred chelator used in the conjugated binding molecules of the present disclosure is restrained complexing agent (RESCA). RESCA was previously described in F. Cleeren et al. Theranostics. 2017; 7(11): 2924-2939 (which is incorporated by reference to the extent allowed), which describes a new radiolabeling method, that enables labeling with Al¹⁸F at room temperature using a new restrained complexing agent (RESCA). Although it was speculated that this could be used for new fluorine-18 labeled protein-based radiotracers, this has not become reality. Discouraging experimental results from rodent studies have greatly diminished the interest for using RESCA, where instability and defluorination has been observed in mouse and rat in vivo, and the DOTA and NOTA chelators as described above has instead been used.

However, in experiments performed by the current inventors, where PDGF-Rβ binding molecules were functionalized with several different moieties to enable radiolabeling and PET imaging of PDGF-Rβ, it was surprisingly found that the PDGF-Rβ binding molecules functionalized with RESCA displayed markedly increased contrast in several tissues with PDGF-Rβ expression, compared with other radiolabeling strategies. In particular, it has been shown that the choice of PDGF-Rβ targeting binding polypeptides as described above, especially Z-variant Z09591, functionalization strategy (RESCA functionalization) and choice of radiolabel (Al¹⁸F), gave unexpected synergies to generate new radiopharmaceuticals with excellent characteristics to provide high contrast PET images of fibrogenesis, as shown in the example section below. These results may be due to several beneficial factors, such as rapid clearance of the non-specific uptake, combined with retained affinity to the target. Also, that RESCA with Al¹⁸F is hydrophilic could decrease non-specific retention, and free ¹⁸F would not be retained in e.g., liver and spleen, while free ⁶⁸Ga will form colloids that may be retained in liver and spleen. Different interactions with plasma may be an additional factor, where RESCA-Z09591 disappears from the blood faster. In an example, a PDGF-Rβ binding polypeptide was modified with a C-terminal Cysteine, where RESCA was joined to the C-terminal Cysteine by malemide chemistry, which gave a construct of the form: polypeptide-linker-chelator-radionuclide. In a specific embodiment, where the polypeptide is Z-variant Z09591 and the radionuclide is Al¹⁸F, the conjugated binding molecule have the structure (construct): Al¹⁸F-RESCA-Z09591.

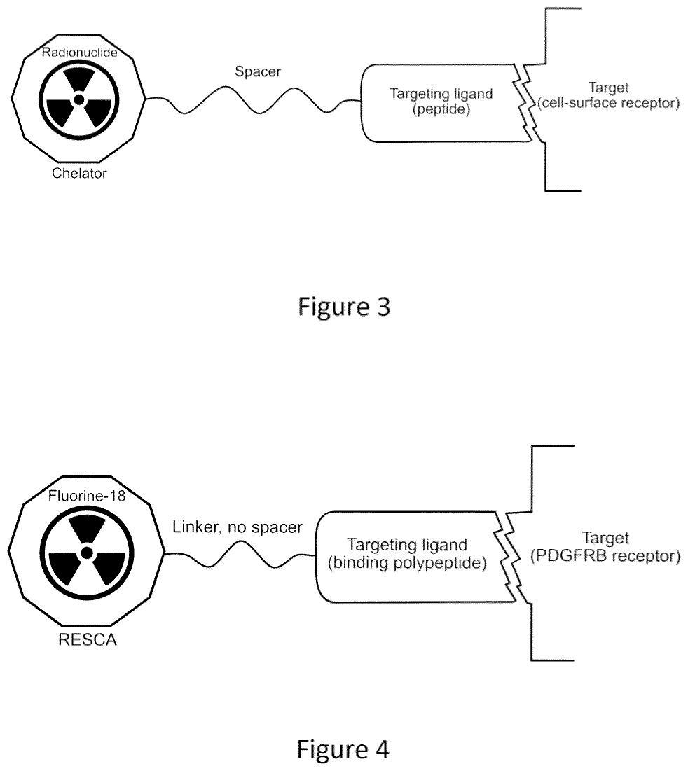

As illustrated in FIG. 3, the typical design of a radiotracer comprises a targeting ligand which bind the target, a spacer, and a chelator comprising the radionuclide, wherein the targeting ligand should have high binding affinity and specificity to target, suitable pharmacokinetics and low immunogenicity and toxicity, the radionuclide must have favorable nuclear characteristics; suitable half-life, high positron abundance, and low positron energy, and the chelator should hold radionuclides with high stability under physiological conditions, and where radiolabeling strategies should preferably be easy to set up. The spacer may be used as a linker, but also to introduce distance between the peptide sequence and chelators, and also to increase hydrophilicity and stability.

In FIG. 4 is illustrated a preferred embodiment of the invention, where the radiotracer, the conjugated binding molecule, comprises a targeting ligand/binding polypeptide as defined in the present disclosure, comprising a 7 kDa peptide scaffold, where the binding polypeptide is raised towards extracellular PDGF-Rβ, refold after denaturation, has a fast distribution and clearance, good tissue penetration and clinical experience. The radionuclide comprises Fluorine-18 (chelation is performed after coupling to an aluminum ion to attain Al¹⁸F), which is the most frequently used radioisotope in PET, and has good nuclear characteristics (97% β+ decay, 109.7 min half-life, 635 keV positron energy), versatile chemistry and high spatial resolution. The chelator is RESCA, a new chelator for Al¹⁸F radiolabeling. In the present construct, a spacer is not required (only a non-spacer “linker”) as the binding site is located far from chelator, on different helices. The linker is in this case the malemide coupling, which is the link between the polypeptide and the RESCA chelator. However, in other embodiments other linkers, including spacer type linkers, may be used.

The conjugated binding molecules may thus be used in Position Emission Tomography (PET) imaging, as a radioactively labelled contrast agent, which may be referred to as a radiotracer or PET tracer. The conjugated binding molecules may also be used in the assessment, detection or diagnosis of fibrotic diseases, including pulmonary fibrosis, liver cirrhosis, scleroderma, glomerulosclerosis, tumor stroma, and cardiac fibrosis, fibrosis degree and/or active fibrogenesis.

In some embodiments, an in vivo method for detecting activated fibroblast and/or mesenchymal stromal cells in a subject, and/or detecting expression of platelet derived growth factor receptor beta (PDGF-Rβ), the method comprising:
(i) administering one or more conjugated binding molecule of according to the present disclosure to the subject; and
(ii) detecting that the one or more conjugated binding molecule has bound fibroblast cells and/or mesenchymal stromal cells.

Bound conjugated binding molecules indicate expression of PDGF-Rβ, which may be used for detecting activated fibroblast cells. Also, active fibrogenesis may be detected by bound conjugated binding molecules. Bound fibroblast cells are determined as being “activated”. It has been shown that fibroblast have to be activated to proliferate and migrate, in processes and pathophysiological conditions, such as wound healing and fibrosis. Activation of fibroblasts typically occurs through four distinct mechanisms: stimulation by growth factors (“auto- and paracrine”), by direct cell-cell contacts, by extracellular matrix via integrins, and by environmental conditions such as hyperglycemia or hypoxia in renal disease. When becoming activated, they express PDGF-Rβ, which resting fibroblasts do not. Hence, detecting that a conjugated binding molecule has bound a fibroblast cell indicates that the cell is activated. The same applies to mesenchymal stromal cells.

The conjugated binding molecules may be administered via one or more routes of administration using one or more of a variety of methods known in the art. As will be appreciated by the skilled artisan, the route and/or mode of administration will vary depending upon the desired results.

Preferred routes of administration include intravenous, intramuscular, intradermal, intraperitoneal, subcutaneous, spinal or other parenteral routes of administration, for example by injection or infusion.

The content of this disclosure thus enables treatment, imaging and diagnosis of disorders linked to PDGF-Rβ expression, such as fibrosis, by administering conjugated binding molecules of the invention. In the drawings and specification, there have been disclosed exemplary aspects of the disclosure. However, many variations and modifications can be made to these aspects without substantially departing from the principles of the present disclosure. Thus, the disclosure should be regarded as illustrative rather than restrictive, and not as being limited to the particular aspects discussed above. Accordingly, although specific terms are employed, they are used in a generic and descriptive sense only and not for purposes of limitation.

The description of the example embodiments provided herein are presented for purposes of illustration. The description is not intended to be exhaustive or to limit example embodiments to the precise form disclosed, and modifications and variations are possible in light of the above teachings or may be acquired from practice of various alternatives to the provided embodiments. The examples discussed herein were chosen and described in order to explain the principles and the nature of various example embodiments and its practical application to enable one skilled in the art to utilize the example embodiments in various manners and with various modifications as are suited to the particular use contemplated. The features of the embodiments described herein may be combined in all possible combinations of methods, products, and systems. It should be appreciated that the example embodiments presented herein may be practiced in any combination with each other. It should also be noted that the word “comprising” does not necessarily exclude the presence of other elements or steps than those listed and the words “a” or “an” preceding an element do not exclude the presence of a plurality of such elements. It should further be noted that any reference signs do not limit the scope of the claims, that the example embodiments may be realized in the broadest sense of the claims.

### Examples

In the following experimental section, a construct of the conjugated binding molecule has been used in which the binding molecule is a Z-molecule according to SEQ ID NO: 237 (also referred to as Z09591), linked by malemide chemistry to a RESCA chelator carrying the radioisotope ¹⁸F, coupled to an aluminum ion as Al¹⁸F.

### Example 1. Structure of RESCA

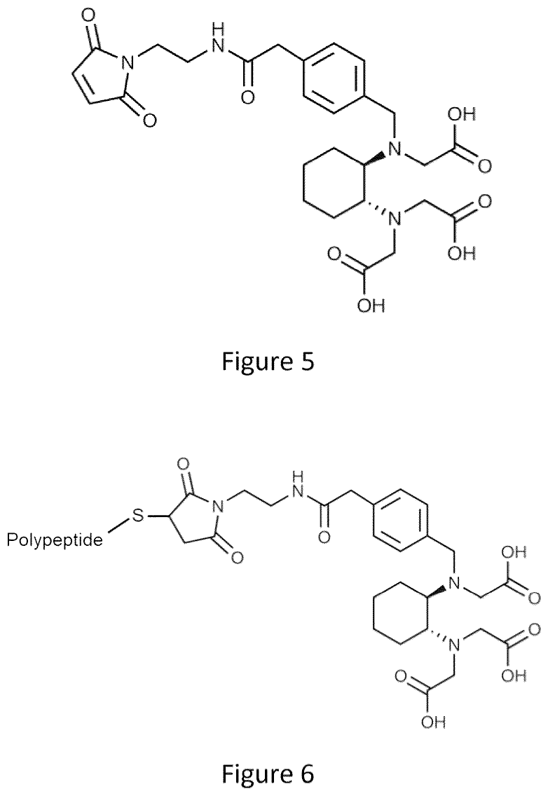

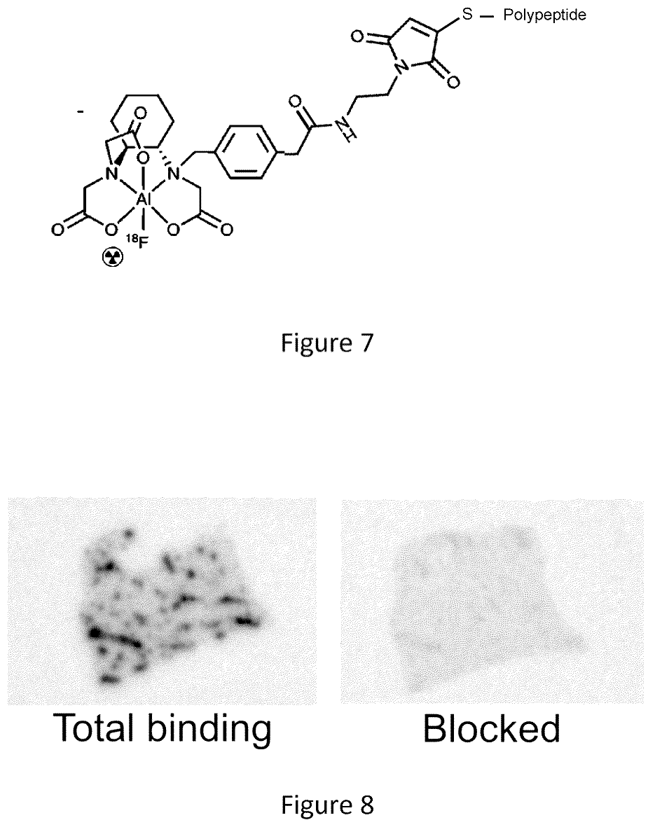

In the following examples, a RESCA chelator has been used, as discussed above. FIG. 5 shows the structure of the RESCA chelator with a malemide group. Further, FIG. 6 shows RESCA chelator conjugated to a polypeptide via malemide chemistry, and FIG. 7 shows AI[18F] coordinated in a RESCA chelator, conjugated to a polypeptide via malemide chemistry.

### Example 2. Characterization of the AI[18F]RESCA-Labeled Z-Molecule by Surface Plasmon Resonance

Aims: This example describes the determination of the RESCA-labeled Z-molecule targeting PDGFRB in vitro by Surface Plasmon Resonance.

### Methods: Surface Plasmon Resonance Studies

The affinities of RESCA-Z to the human PDGFR-β and -α (rhPDGF Rβ, rhPDGF Ra) and murine PDGFRβ (rmPDGF Rβ) were analyzed using a Biacore® T200 SPR instrument (GE Healthcare). The three target proteins were diluted to 10 μg/ml in an acidic immobilization buffer consisting of 10 mM Acetate pH 5.0 for the human receptors and pH 4.5 for the murine receptor. The receptors rhPDGF Rβ, rhPDGF Rα, and rmPDGF Rβ were immobilized by amine coupling in separate flow cells of a CM-5 sensor chip (GE Healthcare). To reduce mass-transport limitations, the immobilization density was maintained low (400-600RU). The analyte, RESCA-Z, was diluted in HBS-EP buffer (10 mM HEPES, 0.15 mM NaCl, 3 mM EDTA, and 0.05% v/v Surfactant P20, pH 7.4). A multi-cycle kinetics method with a broad concentration range of 3000 nM, diluted in a series of three times to 0.15 nM was used to study the affinity of RESCA-Z binding to proteins. This was sufficient to achieve saturation. The interaction between the sample and the target was broken after injection with a 30 s injection of 10 mM NaOH and 1 M NaCl. To allow the surface to stabilize, it was left in the HBS-EP buffer flow for 480 s before the start of a new sample cycle. Each sample was injected at a constant flow rate of 30 μl/min over the receptor surface for 60 s and was left to dissociate for 180 s.

Results: The equilibrium dissociation constant (K_{D}) of RESCA-Z for human PDGFRβ was 0.33±0.03 nM (n=6) and for murine PDGFRβ 2.4±0.5 nM. No measurable interaction was observed towards human PDGFRa at the concentrations used here (up to 3 μM).

### Example 3, Characterization of the AI[18F]RESCA-Labeled Z-Molecule In Vitro in Fibrotic Tissue

Aims: This example describes the evaluation of the AI[18F]RESCA-labeled Z-molecule in vitro in human fibrotic liver tissue.

### Methods: In Vitro Autoradiography Studies

In vitro autoradiography was performed on a frozen human liver biopsy from a patient with liver fibrosis. The sections were cooled to room temperature. Then pre-incubated in 150 mL of a solution of phosphate-buffered saline (PBS) and 1% bovine serum albumin (BSA) at room temperature for 15 min. To study the specificity, a blocking study was performed by adding 2 μM of unlabeled Cys-Z-molecule to the previous solution. After 15 min, AI[18F]RESCA-Z molecule was added to the solution at a concentration of 5 nM (0.1 MBq/mL), and incubated for 60 min. Two washes in cold PBS/BSA 1% and one dip in MQ water were realized to remove the unbound radiotracer. The sections were then dried at 37° C. Calibration standards were created by adding a drop of 10 uL of the incubation solution containing the radiotracer onto absorbent paper. The slides and standards were exposed to a phosphor-imaging plate (BAS-MS, Fuji-film) overnight and scanned by a Phosphor imager (Amersham Typhoon FLA 9500 Phospor Imager, GE). Images were analyzed using ImageJ software (National Institutes of Health, US). PDGFRβ immunostaining and staining for collagen were separately performed on biopsies from the same liver to verify fibrosis degree.

Results: In the fibrotic liver incubated with the radiotracer alone, strong heterogeneous uptake was observed, while this uptake could not be seen in the biopsy incubated with Cys-Z, as illustrated in FIG. 8. Radioactivity uptake correlated with collagen deposition and PDGFRβ expression.

### Example 4, Characterization of the AI[18F]RESCA-Labeled Z-Molecule In Vivo in Xenograft Tumors

Aims: This example describes the evaluation of the AI[18F]RESCA-labeled Z-molecule PDGFRB in vivo in mice bearing xenograft from cells expressing PDGFRβ, in comparison with three other analogues using different chelators.

### Methods:

### Cell Culture

The PDGFRβ expressing cell line U-87 (ATCC) was cultured in Eagle's minimum essential medium (ATCC) and in RPMI-1640 medium (Biowest) respectively, with 10% fetal bovine serum (Merck, Germany), and 1% Penicillin-Streptomycin (Biochrom, Berlin, Germany). U-87 cells at a density of 5×10⁶ were suspended in PBS to be injected into mice for the xenograft model.

### U-87 Xenograft Mice Model

40 female outbred immunodeficient BALB/c nu/nu mice (weight: 19±1 g) were inoculated with U-87 xenografts. Tumors were grafted by subcutaneous injection of U-87 MG cells in the hind leg (2 million cells/mouse, implanted 28 days before the experiment in a volume of 100 μl).

### In Vivo Imaging and Ex Vivo Biodistribution in Mice Bearing U-87 Xenografts

Comparative biodistribution and PET/CT imaging were performed on mice bearing U87 xenografts. The average tumor size at the time of the experiments was 0.07±0.006 g. These mice were separated into 8 groups of 4 mice for ex vivo biodistribution. The remaining 8 mice were used for PET/MRI imaging.

For biodistribution, 4 groups of 4 mice were intravenously injected into the tail vein with either [⁶⁸Ga]DOTA-Z (950 kBq, 0.32 μg, 100 μL), [⁶⁸Ga]NOTA-Z, (980 kBq, 0.36 μg, 100 μL), [¹⁸F]TCO-Z (620 kBq, 0.35 μg, 100 μL), or Al[¹⁸F]RESCA-Z (700 kBq, 0.27 μg, 100 μL). In order to evaluate binding specificity, for each radiotracer ([⁶⁸Ga]DOTA-Z, [⁶⁸Ga]NOTA-Z, [¹⁸F]TCO-Z, and Al[¹⁸F]RESCA-Z) a group of 4 mice was co-injected with excess (200 μg) unlabeled Cys-Z molecule.

For PET/CT imaging, 4 mice were intravenously injected into the tail vein with [⁶⁸Ga]DOTA-Z (6 MBq), [⁶⁸Ga]NOTA-Z (6 MBq), [¹⁸F]TCO-Z (9 MBq), or Al[¹⁸F]RESCA-Z (9 MBq), respectively.

Binding specificity was evaluated in 4 additional mice by co-injecting excess (200 μg) unlabeled Cys-Z molecule.

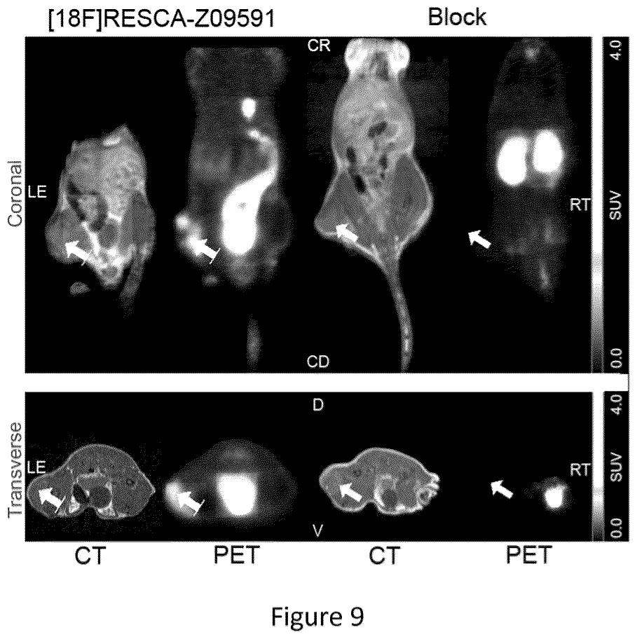

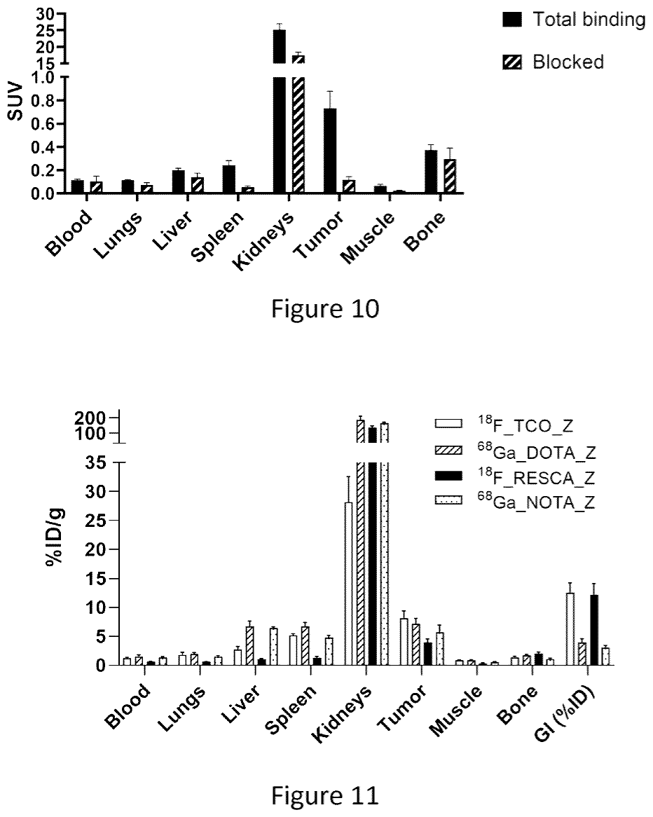

Results: Al[¹⁸F]RESCA-Z PET imaging on mice bearing U-87 xenograft, allowed a clear visualization of the tumor expressing the target (FIG. 9). The uptake in tumors seen both in vivo through PET imaging and ex vivo by biodistribution could be blocked by Cys-Z molecule co-injection (FIG. 10).

Biodistribution of [¹⁸F]TCO-Z, [⁶⁸Ga]DOTA-Z, Al[¹⁸F]RESCA-Z, and [⁶⁸Ga]NOTA-Z in female Balb/c nu/nu mice bearing U-87 MG xenografts at 1 h pi (total injected mass of 0.3 μg) demonstrated tumor visualization with all evaluated tracer analogues (FIG. 11). AI[¹⁸F]RESCA-Z demonstrated lower background in most tissues compared to the three other analogues. Data are presented as an average value and standard deviation for four mice.

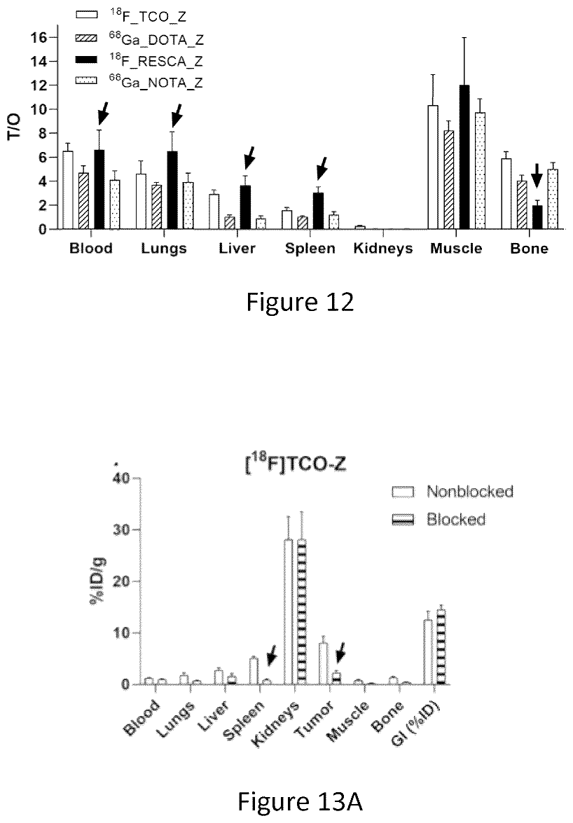

The Tumor-to-organ ratios of [¹⁸F]TCO-Z, [⁶⁸Ga]DOTA-Z, Al[¹⁸F]RESCA-Z, and [⁶⁸Ga]NOTA-Z in female Balb/c nu/nu mice with subcutaneous U-87 MG xenografts at 1 h pi demonstrated higher image contrast for Al[¹⁸F]RESCA-Z in several tissues (FIG. 12).

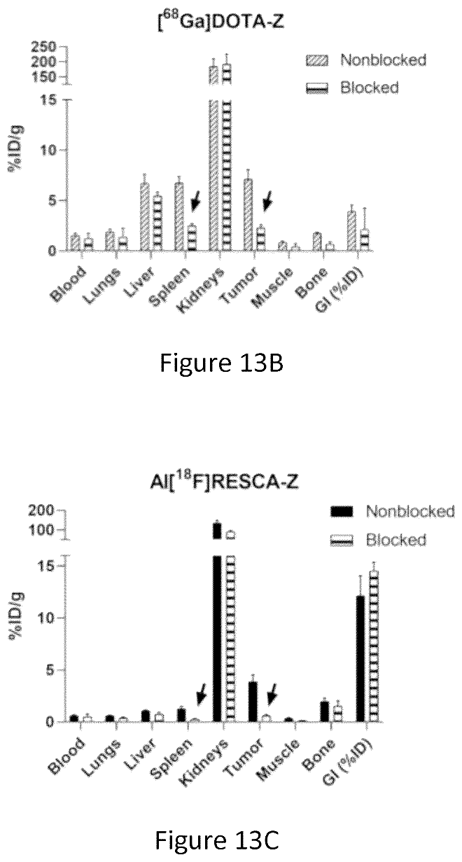

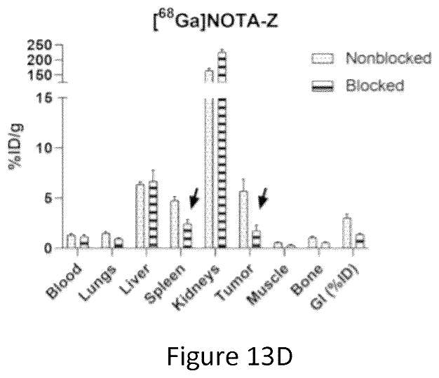

In vivo binding specificity of (A) [¹⁸F]TCO-Z, (B) [⁶⁸Ga]DOTA-Z, (C) Al[¹⁸F]RESCA-Z, and (D) [⁶⁸Ga]NOTA-Z was evaluated in female Balb/c nu/nu mice with subcutaneous U-87 MG xenografts at 1 h pi (FIGS. 13A-13D). The total injected mass of radio conjugate was 0.3 μg. All animals in the blocked group were co-injected with 200 μg of non-labeled peptide. The uptake in tumors seen both in vivo through PET imaging and ex vivo by biodistribution could be blocked by Cys-Z molecule co-injection for all analogues including AI[¹⁸F]RESCA-Z.

In conclusion, AI[18F]RESCA-Z is able to selectively image PDGFRβ in vivo with a lower background than other tracer analogues and therefore with improved image contrast.

### Example 5, In Vivo Imaging with AI[18F]RESCA-Labeled Z-Molecule In Vivo in a Liver Fibrosis Model

Aims: This example describes the use of AI[18F]RESCA-labeled Z-molecule for in vivo PET imaging of liver fibrogenesis.

### Methods:

### CCl4 Mice Model of Liver Fibrosis

C57BL/6J female mice (n=14) received a carbon tetrachloride (CCl4) (Sigma) treatment. Intraperitoneal CCL4 injections at a dose of 0.5 mg/g of body weight in a 1:4 CCl4: corn oil mixture, three times a week for six weeks were performed on the mice. Healthy C57BL/6J female mice were included in the study as a control group n=15. In the CCL4-treated mice group, eight were injected with Al[¹⁸F]RESCA-Z molecule alone, while six were co-injected with a solution containing Al[¹⁸F]RESCA-Z molecule and Cys-Z molecule in excess (1 mg/kg) in order to saturate the target PDGRFβ (blocking study). In the healthy mice group, eight were injected with tracer alone, seven were co-injected with Cys-Z molecule for blocking. All mice were used for ex vivo biodistribution, and ex vivo ARGs. A subset of animals were used for PET-MRI scans (details provided below).

### In Vivo Imaging in the CC4 Model

Three CC4 mice and three healthy mice were injected with 2MBq (1.5 μg peptide) of AI[18F]RESCA-Z molecule alone, or co-injected with 1 mg/kg of Cys-Z molecule. After one hour, the mice were euthanized, placed in a prone position on the scanner bed and a static whole-body PET-MRI scan (Mediso Medical ImagingSystems) was performed. After the PET-MRI scans, the mice were dissected and the organs were harvested for ex vivo biodistribution. The liver, spleen and piece of muscle were used for ex vivo autoradiography. The software PMOD 4.0 (PMOD Technologies) was used to analyze the PET-MRI images.

### Ex Vivo Biodistribution

Eight mice CCl4 mice and eight healthy mice were injected with 2 MBq (1.5 μg peptide) of AI[18F]RESCA-Z molecule alone. In addition, six CCl4 mice, and seven healthy mice received a co-injection of 1 mg/kg of Cys-Z molecule. One hour post-injection the mice were euthanized 1 h and an ex vivo biodistribution was performed as described in example 4.

### Ex Vivo Autoradiography

The liver, leg muscle, and spleen from the biodistribution study were snap-frozen and embedded in OCT. Then they were sectioned at 20 μm in consecutive triplicates using a cryostat microtome (Micron HM560, Germany) and mounted on Superfrost Plus glass slides (Menzel-Glsser). The slides were then exposed to a phosphor-imaging plate (BAS-MS, Fuji-film) and scanned by a Phosphor imager (Amersham Typhoon FLA 9500 Phospor Imager, GE).

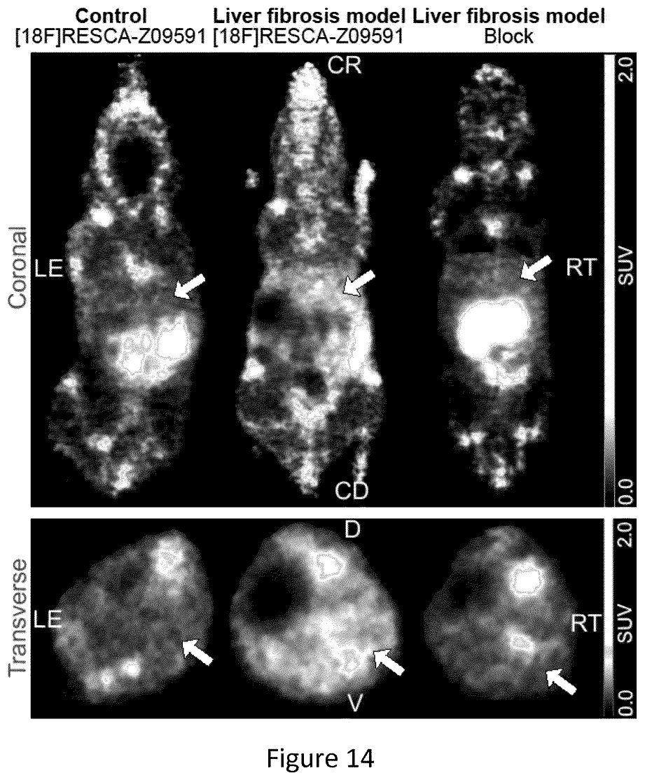

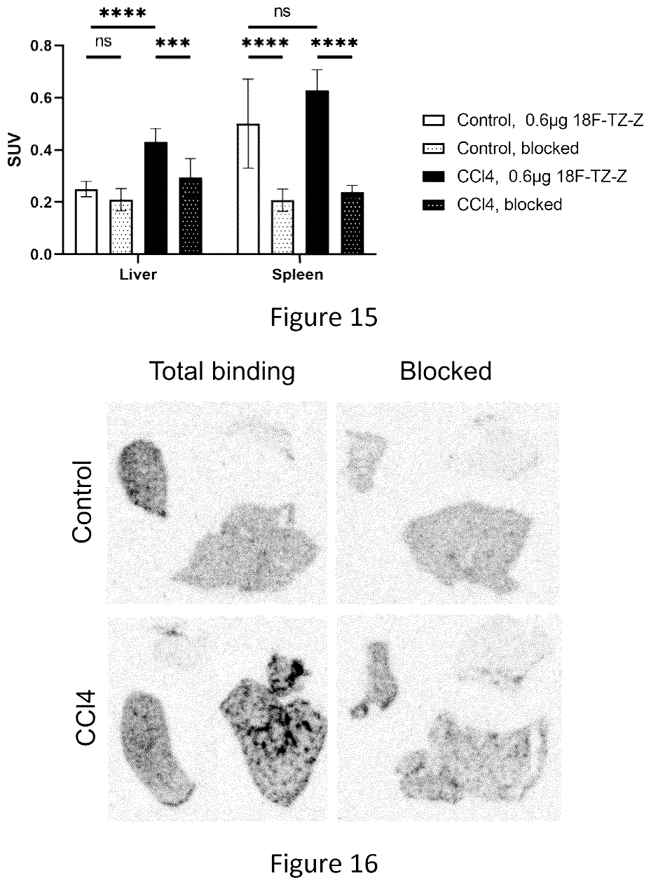

Results and conclusions: AI[18F]RESCA-Z molecule binding was low in the liver of healthy mice and mice blocked with Cys-Z injections. The binding was elevated in the fibrotic liver of mice injected with AI[18F]RESCA-Z molecule alone (FIGS. 14 and 15). Ex vivo autoradiography demonstrated that binding occurred in fibrotic lesions positive for PDGRFβ (FIG. 16). This demonstrates that AI[18F]RESCA-Z molecule selectively binds to PDGRFβ in the vicinity of fibrous scars.

1. A conjugated binding molecule comprising:
i) at least one binding polypeptide, the binding polypeptide being a platelet derived growth factor receptor beta (PDGF-Rβ) binding polypeptide; and
ii) at least one agent, the at least one agent being indirectly joined to the binding polypeptide via a linker in the form of a chelator, wherein the at least one agent comprises or consists of a radionuclide and wherein the chelator is Restrained Complexing Agent (RESCA).

2. The conjugated binding molecule according to claim 1, wherein the radionuclide is Fluorine-18 conjugated to Aluminum in the form of Aluminum-Fluorine-18 (Al¹⁸F).

3. The conjugated binding molecule according to claim 1, wherein the at least one PDGF-Rβ binding polypeptide comprises a platelet derived growth factor receptor beta binding motif, PBM, which motif consists of the amino acid sequence selected from
i) EX₂X₃X₄AAX₇EID X₁₁LPNLX₁₆X₁₇X₁₈QW NAFIX₂₅X₂₆LX₂₈X₂₉,
wherein, independently of each other,
X₂ is selected from L, R and I;
X₃ is selected from R, I, L, V, K, Q, S, H, and A;
X₄ is selected from A, R, N, D, Q, E, H, K, M, S, T, W, F and V;
X₇ is selected from A, R, D, Q, E, G, K and S;
X₁₁ is selected from A, R, N, D, E, G, K, S, T and Q;
X₁₆ is selected from N and T;
X₁₇ is selected from R and K;
X₁₃ is selected from A, R, N, D, C, Q, E, G, L, K, M, S, T, W and V;
X₂₅ is selected from K, R, Q, H, S, G and A;
X₂₆ is selected from S and K;
X₂₈ is selected from V, R, I, L and A;
X₂₉ is selected from D and K; and
ii) an amino acid sequence which has at least 89% identity to the sequence defined in i), and wherein
the PDGF-Rβ-binding polypeptide binds to PDGF-Rβ such that the K_{D} value of the interaction is at most 1×10⁻⁶ M.

4. The conjugated binding molecule according to claim 3, wherein said PDGF-Rβ-binding motif forms part of a three-helix bundle protein domain, and wherein in which said PDGF-Rβ-binding motif essentially forms part of two alpha helices with an interconnecting loop, within said three-helix bundle protein domain.

5. The conjugated binding molecule according to claim 3, wherein at least one PDGF-Rβ binding polypeptide comprises an amino acid sequence selected from:
VDNKFNK-[PBM]-DPSQSANLLAEAKKLNDAQAPK; and
AENKFNK-[PBM]-DPSQSANLLAEAKKLNDAQAPKC.

6. The conjugated binding molecule according to claim 3, wherein at least one PDGF-Rβ binding polypeptide comprises an amino acid sequence selected from SEQ ID NO: 4-351

7. The conjugated binding molecule according to claim 3, wherein at least one PDGF-Rβ binding polypeptide is a Z-molecule comprising the amino acid sequence:
(SEQ ID NO: 237)
AENKFNKELIEAAAEIDALPNLNRRQWNAFIKSLVDDPSQSANLLAEAK
KLNDAQAPKC.

8. The conjugated binding molecule according to claim 7, wherein the binding polypeptide is the Z-molecule comprising the amino acid sequence SEQ ID NO: 237 and the radionuclide is Al¹⁸F, and wherein the conjugated binding molecule has the construct: Al¹⁸F-RESCA-Z.

9. The conjugated binding molecule according to claim 1, wherein the chelator is joined to the binding polypeptide via a linker.

10. A conjugated binding molecule according to claim 1, for use in Position Emission Tomography (PET) imaging.

11. A conjugated binding molecule according to claim 1, for use in the assessment of fibrosis, fibrosis degree and/or active fibrogenesis.

12. An in vivo method for detecting activated fibroblast cells and/or activated mesenchymal stromal cells in a subject by detecting expression of platelet derived growth factor receptor beta (PDGF-Rβ), the method comprising:
(i) administering one or more conjugated binding molecules of claim 1 to the subject; and
(ii) detecting that the one or more conjugated binding molecule has bound fibroblast cells and/or mesenchymal stromal cells, wherein the bound fibroblast cells and/or bound mesenchymal stromal cells are determined as being activated.
